(19) United States
(12) Patent Application Publication
(10) Pub. No.: US 2025/0160275 A1
(43) Pub. Date: May 22, 2025

(54) COMPOUNDS FOR MODIFYING MEIOTIC RECOMBINATION AND METHODS RELATING THERETO

(71) Applicant: The University of North Carolina at Chapel Hill, Chapel Hill, NC (US)

(72) Inventor: Gregory Paul Copenhaver, Chapel Hill, NC (US)

(21) Appl. No.: 18/839,089

(22) PCT Filed: Feb. 24, 2023
(86) PCT No.: PCT/US2023/063204

§ 371 Date: Aug. 16, 2024

Related U.S. Application Data
(60) Provisional application No. 63/268,515, filed on Feb. 25, 2022.

(51) Int. Cl. A01H 1/06

(52) U.S. Cl. CPC A01H 1/06

(57) ABSTRACT

Described herein are compounds for modifying meiotic recombination in a plant. In addition, described herein are methods of modifying meiotic recombination in a plant, including methods that can reduce linkage drag in a plant, increase recombination in a cold region of the genome of a plant, and/or reduce the number of backcross generations in a plant breeding method.

### FIELD

This invention relates to compounds for modifying meiotic recombination in a plant and to methods relating thereto such as methods of modifying meiotic recombination in a plant.

### BACKGROUND OF THE INVENTION

Meiosis is the specialized form of cell division, common to all sexually reproducing organisms, that reduces the genomic compliment by half in preparation for fertilization, and produces gametes (sperm or eggs). During meiosis, most organisms shuffle genetic information between the homologous chromosomes they inherited from their parents in a process called recombination. Meiotic recombination produces novel combinations of the versions of the genes (alleles) carried on each parental chromosome which in turn causes genetic and phenotypic variation. Generating and selecting desirable variation is the basis for all commercial plant breeding.

During breeding programs there are times when it is beneficial to have high levels of recombination so that desirable traits can be separated from undesirable ones—this is often referred to as avoiding “linkage drag”. At other times it is beneficial to have low levels of recombination so that multiple linked desirable traits can be maintained as a group. Breeders typically approach these problems in two ways. They can screen large populations that are “segregating” the traits of interest to identify the individuals that express the desired trait(s) but lack the undesirable trait(s). Segregating populations are created by crossing parents that have different genotypes at one or more loci in their genome (genetic polymorphisms). The resulting F1 (first filial) progeny are heterozygous at the polymorphic loci. F1 progeny are then crossed with one another or allowed to self-fertilize to generate F2 (second filial generation) progeny; or backcrossed to one of the parents to generate BC1 (first backcross generation) progeny; or outcrossed to another line to create outcrossed progeny. The meiotic recombination that occurs during the production of gametes by the F1 individuals generates new combinations of alleles in the subsequent progeny generations. Because the new combinations of alleles differ between F2, or BC1 or outcrossed individuals those generations are said to be “segregating” the alleles. Breeders apply their selection schemes to these segregating populations, but this is expensive, laborious, time consuming and difficult to scale. Alternatively, they can use genetic engineering to influence the genes than regulate meiotic recombination, but this approach has consumer acceptance issues. Accordingly, new methods that can modify meiotic recombination would be advantageous.

### SUMMARY OF THE INVENTION

A first aspect of the present invention is directed to a method of modifying meiotic recombination in a plant, the method comprising: applying a compound that modulates meiotic recombination to the plant, thereby modifying meiotic recombination in the plant.

A further aspect of the present invention is directed to a method of reducing linkage drag in a plant, increasing recombination, including increasing recombination in a cold region (a region with low recombination), of a plant genome, and/or reducing the number of backcross generations in a plant breeding method, the method comprising: applying a compound that modulates meiotic recombination to a plant, thereby reducing linkage drag in the plant, increasing recombination in a cold region of the genome of the plant, and/or reducing the number of backcross generations in a plant breeding method including the plant.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of 10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

According to embodiments of the present invention provided are compounds that can modulate (e.g., increase or decrease) meiotic recombination in a plant. In addition, provided according to embodiments of the present invention is a method of modifying meiotic recombination in a plant, the method comprising applying to the plant a compound that modulates meiotic recombination, thereby modifying meiotic recombination in the plant. In some embodiments, a method of the present invention comprises reducing linkage drag in a plant, increasing recombination, including increasing recombination in a cold region (a region of low recombination), in the genome a plant, and/or reducing the number of backcross generations in a plant breeding method including a plant, the method comprising applying a compound that modulates meiotic recombination to the plant to thereby reduce linkage drag in the plant, increase recombination in the genome of the plant, (e.g., increase recombination in a cold region of the genome of the plant), and/or reduce or eliminate the number of backcross generations in a plant breeding method including the plant.

In some embodiments, a method of the present invention is devoid of genetic engineering to modify meiotic recombination in the plant. A method of the present invention may not rely on and/or involve stable transgenic changes to a plant's genome. In some embodiments, a method of the present invention may accelerate genetic gain in a plant breeding method and/or decrease breeding time, optionally in a non-genetically modified fashion.

A method of the present invention and/or a compound that modulates meiotic recombination of the present invention may increase the frequency of meiotic recombination events that occur in the genome of a plant. Meiotic recombination is initiated by double strand breaks (DSBs) in the DNA (e.g., DSBs in chromosomes) of plants. During meiotic recombination DSBs are repaired. DSB repair can be mediated by multiple molecular pathways including: “double strand break repair’ (DSBR), “synthesis dependent strand annealing” (SDSA), MUS81-dependent repair, YENI/GEN1-dependent repair, double-holiday junction dissolution, non-homologous end joining (NHEJ), break-induced repair (BIR), one-sided DSB repair, gap repair, and sister-chromatid exchange (SCE). These pathways can result in crossovers (CO; reciprocal exchange of DNA between homologous chromosomes), non-crossover (NCO; repair without reciprocal exchange of DNA between homologous chromosomes), or non-homologous rearrangements (for example translocations or inversions). All of these recombination products can also be accompanied by “gene conversion” (GC) which is the conversion of sequence information from allele to another. Among these products of meiotic recombination, the frequency of meiotic crossover events is a limiting factor in breeding programs. Thus, increasing the frequency of meiotic recombination in a plant, which can include increasing the number of crossovers that occur during meiosis in a cell of the plant, may reduce linkage drag, increase recombination in the genome of the plant (e.g., increase recombination in a cold region of the genome of the plant), and/or reduce or eliminate the number of backcross generations in a plant breeding method including the plant. In some embodiments, a method of the present invention decreases the frequency of meiotic recombination in a plant (e.g., decreases the number of crossovers that occur during meiosis in a cell of the plant), which may allow for two or more (e.g., 2, 3, 4, or more) desirable traits that are optionally linked to be maintained as a group. An increase or decrease in the frequency of meiotic recombination in a plant can be compared to the frequency of meiotic recombination in a control plant and/or a parent plant. A “control plant” as used herein refers to a plant of the same species, breeding line, variety, and/or cultivar as the plant to which a compound that modulates meiotic recombination is applied, but a compound that modulates meiotic recombination of the present invention is not applied to the control plant. In some embodiments, the method comprises comparing a plant of the present invention and a control plant that are grown under the same growth conditions, e.g., the same environmental conditions (e.g., soil, hydration, light, heat, and/or nutrient conditions, and/or the like). In some embodiments, an increase or decrease in the frequency of meiotic recombination is determined by comparing the frequency of meiotic recombination in the gametes of a parent plant and in the gametes of a progeny plant that is the progeny of the parent plant and wherein a compound that modulates meiotic recombination is applied to the progeny plant, optionally wherein the parent plant and progeny plant are grown under the same growth conditions. In some embodiments, an increase or decrease in the frequency of meiotic recombination is determined by comparing the frequency of meiotic recombination in the gametes of a first progeny plant that is the progeny of parental plants wherein a compound that modulates meiotic recombination is applied to the first progeny plant and the frequency of meiotic recombination in the gametes of a second progeny plant that is the progeny of the same parental plants as the first progeny plant, wherein the second progeny plant has not been contacted with a compound that modulates meiotic recombination, optionally wherein the first and second progeny plants are grown under the same growth conditions.

A method of the present invention may comprise determining an increase or decrease in the frequency of meiotic recombination in a plant. In some embodiments, determining an increase or decrease in the frequency of meiotic recombination in a plant may comprise measuring the frequency of meiotic recombination using a visual fluorescent pollen transgene assay (e.g., a pollen tetrad-based visual assay). In some embodiments, determining an increase or decrease in the frequency of meiotic recombination in a plant may comprise measuring the frequency of meiotic recombination using the polymerase chain reaction (PCR) method to measure the segregation of one or more linked molecular markers (DNA polymorphisms). In some embodiments, determining an increase or decrease in the frequency of meiotic recombination in a plant may comprise measuring the frequency of meiotic recombination using nucleic acid sequencing (e.g., high through-put sequencing and/or next generation sequencing (NGS)). In some embodiments, DNA may be obtained and/or isolated from pollen of a plant contacted with a compound that modulates meiotic recombination and may be used to measure the frequency of meiotic recombination optionally via a PCR method and/or nucleic acid sequencing. In some embodiments, determining an increase or decrease in the frequency of meiotic recombination in a plant may comprise measuring the frequency of meiotic recombination by scoring the segregation loci that express observable phenotypes encoded by one or more linked loci. The GLABROUS1 gene in Arabidopsis thaliana is an example of a locus that can be used as an observable marker. Functional alleles of GLABROUS1 allow trichomes to develop on the leaves and organs while non-functional alleles result in a smooth phenotype. Several such phenotypic markers have been described in the literature. Loci that express observable phenotypes can be native to the host genome or transgenes. In some embodiments, a method of the present invention comprises genotyping, optionally wherein the genotyping comprises sequencing a polynucleotide and/or the genome of a gamete and/or pollen of the plant to which a compound that modulates meiotic recombination is applied; sequencing a polynucleotide and/or the genome of a gamete and/or pollen of the plant to which a compound that modulates meiotic recombination is not applied (e.g., a control and/or parent plant), and comparing the sequences to thereby quantify the frequency of meiotic recombination (e.g., the number of crossovers). In some embodiments, a method of the present invention comprises genotyping, optionally wherein the genotyping comprises using a microarray comprised of two or more (e.g., 2, 3, 4, 5, 10, 15, or more) marker(s) that can be simultaneously assayed by hybridization with genomic DNA from pollen or other tissue of a plant to which a compound that modulates meiotic recombination is applied or its progeny; or hybridization with genomic DNA from pollen or other tissue of a plant to which a compound that modulates meiotic recombination is not applied or its progeny (e.g., a control and/or parent plant), and comparing the hybridization patterns to thereby quantify the frequency of meiotic recombination (e.g., the number of crossovers).

A visual fluorescent pollen transgene assay may be performed as described in Francis et al., Proc Natl Acad Sci USA. 2007; 104 (10): 3913-3918, Modliszewski et al., PLOS Genet, 2018, 14 (5): e1007384, and/or Berchowitz, L. and Copenhaver, G. Nature Protocols, 2008; 3 (1), 41-50, the contents of each of which are incorporated herein by reference in their entirety. In some embodiments, measuring the frequency of meiotic recombination using a visual fluorescent pollen transgene assay comprises providing a plant that comprises a nucleotide sequence encoding one or more (e.g., 1, 2, 3, 4, 5, or more) pollen-expressed fluorescent protein(s) that are different from each other (e.g., each protein fluoresces a different color) and/or a nucleotide sequence encoding one or more (e.g., 1, 2, 3, 4, 5, or more) gamete-expressed fluorescent protein(s) that are different from each other. A visual fluorescent pollen transgene assay can employ linked transgenes encoding differently colored fluorescent proteins that are expressed under the control of a post-meiotic, pollen- and/or gamete specific protomer (e.g., a LAT52 promoter) and, by observing the segregation pattern of the fluorescent proteins in pollen tetrads (e.g., by using fluorescence microscopy), the number of crossovers can be quantified. In some embodiments, the method may comprise observing about 1, 10, 50, 100, 150, or 200 to about 250, 300, 350, 400, 450, 500, or more pollen tetrads to determine the segregation pattern of the fluorescent proteins in pollen tetrads and quantify the number of crossovers.

In some embodiments, a method of the present invention may comprise generating a plant that comprises a fluorescent protein (optionally two or more different fluorescent proteins), optionally by transforming the plant with a nucleotide sequence that encodes the fluorescent protein to provide a plant encoding the fluorescent protein, wherein the location of the nucleotide sequence encoding the fluorescent protein in the genome of the plant allows for the frequency of meiotic recombination to be quantified (e.g., in one or more gamete- and/or pollen-expressed fluorescent protein(s)); selecting seed from the plant comprising the fluorescent protein, wherein the seed comprises the nucleotide sequence encoding the fluorescent protein in its genome; growing the seed into a parent plant; measuring the frequency of meiotic recombination in the parent plant by examining and/or determining the fluorescent signal and/or fluorescent pattern from pollen tetrads produced by the parent plant; generating a progeny plant from the plant, optionally by selfing the parent plant; applying a compound that modulates meiotic recombination to the progeny plant; measuring the frequency of meiotic recombination in the progeny plant by examining and/or determining the fluorescent signal and/or fluorescent pattern from pollen tetrads produced by the progeny plant; and determining whether there is an increase or decrease in the frequency of meiotic recombination by comparing the frequency of meiotic recombination in the parent plant and progeny plant.

In some embodiments, a method of the present invention may comprise generating a plant that comprises a fluorescent protein (optionally two or more different fluorescent proteins), optionally by transforming the plant with a nucleotide sequence that encodes the fluorescent protein provide a plant encoding the fluorescent protein, wherein the location of the nucleotide sequence encoding the fluorescent protein in the genome of the plant allows for the frequency of meiotic recombination to be quantified (e.g., in one or more gamete- and/or pollen-expressed fluorescent protein(s)); selecting a plurality of seeds from the plant comprising the nucleotide sequence encoding the fluorescent protein, wherein each seed of the plurality of seeds comprises the nucleotide sequence encoding the fluorescent protein in its genome; growing the plurality of seeds into a plurality of plants, wherein the plurality of plants comprises a first population of plants and a second population of plants; applying a compound that modulates meiotic recombination to the first population of plants, wherein the compound that modulates meiotic recombination is not applied to the second population of plants; measuring the frequency of meiotic recombination in one or more plants in first population of plants and in one or more plants in the second population of plants by examining and/or determining the fluorescent signal and/or fluorescent pattern from pollen tetrads produced by the respective plant; and determining whether there is an increase or decrease in the frequency of meiotic recombination by comparing the frequency of meiotic recombination in the one or more plants in the first population of plants and the frequency of meiotic recombination in the one or more plants in the second population of plants.

A method of the present invention may modify and/or affect one or more pathways involved in meiotic recombination. In some embodiments, a method of the present invention modifies a pathway involved in double-strand break formation, double-strand break end resection, strand invasion, D-loop formation, D-loop extension, second-end capture, formation of recombination intermediates including Holliday junctions and double-Holliday junctions, DNA synthesis in meiotic recombination, Holliday junction resolution, helicase activity, crossover formation, formation of non-reciprocal exchanges, DNA methylation, post-translational histone modification, distribution of histone variants, heat stress, cold stress, pathogen stress, and/or a pathway as described in Wang, Y. and Copenhaver, G., Annu. Rev. Plant Biol. 2018. 69:577-609, the contents of which are incorporated herein by reference in its entirety. In some embodiments, a method of the present invention may increase expression and/or activity of a nucleic acid and/or protein in a plant and/or may decrease expression and/or activity of a nucleic acid and/or protein in a plant. The nucleic acid and/or protein may be involved in a meiotic recombination pathway. Exemplary nucleic acids and/or proteins encoded thereby whose expression and/or production may be increased or decreased by a method of the present invention include, but are not limited to, SPO11-1, SPO11-2, MTOPVIB, PRD1, PRD2, PRD3, DFO, PCH2, PHS1, MRE11, RAD50, NBS1, RAD51, DMC1, POL2A, RFC1, POLD1, MUS81, MSH4, MSH5, HEI10, MLH, FANCM, FIGL1, Ku70/80, Ku70/80, MRE11, PARP1, RAD51, ATM, H2AX, COMI/SAE2, Ku70/80, and/or PARP1, and/or a protein encoded thereby. Further exemplary proteins whose expression and/or production may be increased or decreased by a method of the present invention include, but are not limited to, a histone deacetylase inhibitor, a DNA methyltransferase (e.g., CMT3), a H3K4me3 demethylase inhibitor (e.g., LSD1), a histone deacetylase inhibitor, an inhibitor of H3K9 methylation (e.g., SDG21 and/or SUVH4/5/6), heat shock protein 90 (HSP90), Bloom syndrome protein (BLM), RECQL4, TOP3A, and/or DNA ligase IV. In some embodiments, a method of the present invention may increase or decrease expression, production, and/or activity of an enzyme in a plant.

Applying the compound that modulates meiotic recombination to the plant comprises contacting (e.g., exogenously contacting) the compound to at least a portion of the plant. In some embodiments, applying the compound that modulates meiotic recombination to the plant comprises spraying (e.g., foliar spraying), drenching, injecting, dipping, soaking, and/or the like the compound that modulates meiotic recombination onto at least a portion of the plant. In some embodiments, the compound that modulates meiotic recombination is present in a composition (e.g., an aqueous composition) and the composition is contacted to the plant (e.g., sprayed, dipped, soaked, injected, drenched, and/or the like onto the plant). In some embodiments, the compound that modulates meiotic recombination is present in a composition (e.g., an aqueous composition) and the composition is contacted to the plant (e.g., sprayed, dipped, soaked, injected, drenched, and/or the like onto the plant) to saturate the plant (e.g., coat the plant with the composition (e.g., liquid composition) to the point of the composition dripping off the plant). In some embodiments, the compound that modulates meiotic recombination is contacted to the plant by contacting soil adjacent to the plant with the compound that modulates meiotic recombination and/or by soil drenching (e.g., drenching the soil around the plant with the compound that modulates meiotic recombination). In some embodiments, a compound that modulates meiotic recombination can be present in a hydroponic solution and/or applied to a plant as a liquid, solid, paste, gel, and/or gas. A compound that modulates meiotic recombination can be taken up by vegetative tissue of a plant such as leaves and/or stems; reproductive tissue of a plant such as flowers, anthers, pollen mother cells, ovaries, ovules, and/or megaspore mother cells; a root; and/or by entry through a stomatal opening.

In some embodiments, a compound that modulates meiotic recombination may act on a cell that is undergoing meiosis or is developmentally fated to undergo meiosis (e.g., a meiocyte). A compound that modulates meiotic recombination may be applied to a plant such that the compound acts on a vegetative cell which then differentiates into a meiocyte. In some embodiments, a compound that modulates meiotic recombination may act on a vegetive cell which produces a signal that is received by the meiocyte and/or acts on a cell that will differentiate into a meiocyte. Signals can be communicated cell-to-cell or across tissues, including from the root to the shoot and/or to a floral tissue. In some embodiments, a compound that modulates meiotic recombination that is applied to a plant may produce a persistent or transient effect. A compound that modulates meiotic recombination may be applied to a plant and may act within a single generation, or it can be applied in one generation and influence two or more (e.g., 2, 3, 4, 5, or more) subsequent generations. Plants have an alternation of generation life cycle, so generations include both gametophyte and sporophyte phases.

In some embodiments, a method of the present invention comprises applying a compound that modulates meiotic recombination to a plant at a time prior to, during, and/or after the transition from the vegetative phase to the reproductive phase of the plant. In some embodiments, a method of the present invention comprises applying a compound that modulates meiotic recombination to a plant during the plant's reproductive phase. In some embodiments, a method of the present invention comprises applying a compound that modulates meiotic recombination to a cell of a plant, wherein the cell is a meiocyte and/or is undergoing meiosis, optionally wherein the cell is a vegetative cell. In some embodiments, a method of the present invention comprises applying a compound that modulates meiotic recombination to the sporophyte generation of a plant to modulate meiotic recombination in the gametophyte generation, and the gametophyte generation may have an increased number of crossovers compared to the gametophyte generation generated in the absence of a method of the present invention and/or the parent generation generated in the absence of a method of the present invention.

A method of the present invention may comprise applying a compound that modulates meiotic recombination to a reproductive part of the plant (e.g., a flower bud, inflorescence, flower, stamen, pistil, etc.). In some embodiments, a method of the present invention comprises applying a compound that modulates meiotic recombination to an aerial part of the plant. In some embodiments, a method of the present invention comprises applying a compound that modulates meiotic recombination to all aerial parts of a plant, optionally wherein the applying comprises contacting the aerial parts of the plant with a composition comprising the compound that modulates meiotic recombination (optionally to saturate the plant with the composition). In some embodiments, a method of the present invention comprises applying a compound that modulates meiotic recombination to all aerial parts of a plant when the plant has an inflorescence, optionally wherein the applying comprises contacting the aerial parts of the plant having the inflorescence with a composition comprising the compound that modulates meiotic recombination (optionally to saturate the plant with the composition). In some embodiments, a method of the present invention comprises applying a compound that modulates meiotic recombination to a root of the plant.

According to embodiments of the present invention, a compound that modulates meiotic recombination may be a compound that mimics temperature (e.g., heat and/or cold) shock in a plant, a compound that mimics pathogen stress in a plant, an allelopathic compound, a compound that inhibits non-homologous end joining (NHEJ) in a plant, a compound that affects helicase activity and/or formation in a plant, a compound that affects an epigenetic mark and/or epigenetic modifier in a plant, and/or a compound that affects a component used in a meiotic recombination pathway in a plant. In some embodiments, the compound that modulates meiotic recombination is selected from the group consisting of: ethyl 4-[(1-hydroxy-2-phenylindol-3-yl)-pyridin-2-ylmethyl]piperazine-1-carboxylate, N-(5-tert-butyl-1H-pyrazol-3-yl)-2-[(3R)-3-propan-2-ylpiperazin-1-yl]-7H-pyrrolo[2,3-d]pyrimidin-4-amine, N-(6-aminohexyl)-5-chloro-1-naphthalenesulfonamide, 2-chloro-10-(3-dimethylaminopropyl) phenothiazine hydrochloride, Z-5-(4-hydroxybenzylidene)-2-imino-1,3-thiazolidin-4-one, 2-[(2R)-2-methylpyrrolidin-2-yl]-1H-benzimidazole-4-carboxamide, 5-[(E)-2-(4-hydroxyphenyl) ethenyl]benzene-1,3-diol, 3-(benzylsulfamoyl)-4-bromo-N-(4-bromophenyl)benzamide, 2-(4-Morpholinyl)-6-(1-thianthrenyl)-4H-Pyran-4-one, an azane, a dichloroplatinum (2+) compound, cisplatin, 2-(pyridin-3-ylmethylidene) indene-1,3-dione, [(1S,2S,4R)-4-[4-[[(1S)-2,3-dihydro-1H-inden-1-yl]amino]pyrrolo[2,3-d]pyrimidin-7-yl]-2-hydroxycyclopentyl]methyl sulfamate, 2-[2-(3,4-dimethoxyphenyl)ethylamino]-6-(3-fluorophenyl)-8H-pyrimido[4,5-d]pyrimidine-5,7-dione, 1-(4-((dimethylamino)methyl)phenyl)-8,9-dihydro-2,7,9a-triazabenzo[cd]azulen-6 (7H)-one, (2E,4E,6R)-7-[4-(dimethylamino)phenyl]-N-hydroxy-4,6-dimethyl-7-oxohepta-2,4-dienamide, 4-[(E)-2-(3,5-dimethoxyphenyl) ethenyl]phenol, 4-amino-N-(4,6-dimethylpyrimidin-2-yl)benzenesulfonamide, (2S)-2-amino-4-ethylsulfanylbutanoic acid, 4-amino-1-[(2R,3R,4S,5R)-3,4-dihydroxy-5-(hydroxymethyl)oxolan-2-yl]-1,3,5-triazin-2-one, sodium butanoate, 5,7-dihydroxy-3-(4-hydroxyphenyl) chromen-4-one, pyridine-2,4-dicarboxylic acid, (1R,2S)-2-phenylcyclopropan-1-amine, 4-amino-1-[(2R,4S,5R)-4-hydroxy-5-(hydroxymethyl)oxolan-2-yl]-1,3,5-triazin-2-one, 1-[(2R,3R,4S,5R)-3,4-dihydroxy-5-(hydroxymethyl)oxolan-2-yl]pyrimidin-2-one, 3-[4-[(1R,2S)-2-aminocyclopropyl]phenyl]phenol, methyl 3-[4-(4-carbamimidoylbenzoyl) piperazine-1-carbonyl]-5-[(4-carbamimidoylpiperazin-1-yl)methyl]benzoate, 2-[[[2-[2-(dimethylamino)ethyl-ethylamino]-2-oxoethyl]amino]methyl]pyridine-4-carboxamide, 2-(4-methylphenyl)-1,2-benzothiazol-3-one, 5-chloro-N-[(E)-[phenyl(pyridin-2-yl)methylidene]amino]pyridin-2-amine, methyl 2-benzamido-1-(3-phenylpropyl)benzimidazole-5-carboxylate, 2-[(2-hydroxynaphthalen-1-yl)methylideneamino]-N-(1-phenylethyl)benzamide, 3-(prop-2-enyldisulfanyl) prop-1-ene, N-(1-benzylpiperidin-4-yl)-6,7-dimethoxy-2-(4-methyl-1,4-diazepan-1-yl) quinazolin-4-amine, 5′-methoxy-6′-(3-pyrrolidin-1-ylpropoxy)spiro[cyclobutane-1,3′-indole]-2′-amine, 2-cyclohexyl-6-methoxy-N-(1-propan-2-ylpiperidin-4-yl)-7-(3-pyrrolidin-1-ylpropoxy) quinazolin-4-amine, 7-[3-(dimethylamino) propoxy]-6-methoxy-2-(4-methyl-1,4-diazepan-1-yl)-N-(1-methylpiperidin-4-yl) quinazolin-4-amine, 14-(hydroxymethyl)-3-[14-(hydroxymethyl)-18-methyl-13,17-dioxo-15,16-dithia-10,12,18-triazapentacyclo[12.2.2.01,12.03,11.04,9]octadeca-4,6,8-trien-3-yl]-18-methyl-15,16-dithia-10,12,18-triazapentacyclo[12.2.2.01,12.03, 11.04,9]octadeca-4,6,8-triene-13,17-dione, 2,3-dichloro-1,4-naphthoquinone, 2,3-dichloro-5,8-dihydroxy-1,4-naphthoquinone, (1S,2R,4S)-1,7,7-trimethylbicyclo[2.2.1]heptan-2-ol, 2-isothiocyanatoethylbenzene, 4-isothiocyanatobutylbenzene, 3-isothiocyanatoprop-1-ene, isothiocyanatoethane, 1-isothiocyanatobutane, 3-isothiocyanato-2-methylprop-1-ene, [(4E,6Z,8S,9S,10E,12S, 13R, 14S, 16R)-13-hydroxy-8, 14, 19-trimethoxy-4, 10, 12, 16-tetramethyl-3,20,22-trioxo-2-azabicyclo[16.3.1]docosa-1 (21),4,6,10, 18-pentaen-9-yl]carbamate, 4-[[(2R,3S,4R,5R)-5-[6-amino-8-[(3,4-dichlorophenyl)methylamino]purin-9-yl]-3,4-dihydroxyoxolan-2-yl]methoxymethyl]benzonitrile, 24-methyl-5,7,18,20-tetraoxa-24-azoniahexacyclo[11.11.0.02,10.04,8.014,22.017,21]tetracosa-1 (24),2,4 (8),9,11,13,15,17 (21),22-nonaene, (4S)-4-prop-1-en-2-ylcyclohexene-1-carbaldehyde, (4R)-1-methyl-4-prop-1-en-2-ylcyclohexene, (1R,4R)-1,7,7-trimethylbicyclo[2.2.1]heptan-2-one, 1,3,3-trimethyl-2-oxabicyclo[2.2.2]octane, 2,6,6-trimethylbicyclo[3.1.1]hept-2-ene, isothiocyanatobenzene, 2-isothiocyanatopropane, 1-isothiocyanato-2-methylpropane, 1-isothiocyanatohexane, 1-isothiocyanato-4-methylsulfinylbutane, 1-isothiocyanato-5-methylsulfinylpentane, 8-[(6-iodo-1,3-benzodioxol-5-yl)sulfanyl]-9-[3-(propan-2-ylamino) propyl]purin-6-amine, [4-[2-carbamoyl-5-[6,6-dimethyl-4-oxo-3-(trifluoromethyl)-5,7-dihydroindazol-1-yl]anilino]cyclohexyl]2-aminoacetate, 3-(2,4-dihydroxy-5-propan-2-ylphenyl)-4-(1-methylindol-5-yl)-1H-1,2,4-triazol-5-one, N-acetyl-5-methoxytryptamine, S-methyl 1,2,3-benzothiadiazole-7-carbothioate, 5-chloro-2-methylpyrazole-3-carboxylic acid, 3-prop-2-enoxy-1,2-benzothiazole 1,1-dioxide, 3-aminobutanoic acid, 1,1-dioxo-1,2-benzothiazol-3-one, 4-hydroxybenzohydrazide, 3-hydroxy-3-(2-oxopropyl)-1H-indol-2-one, 2-[3-[(4-amino-2-methylpyrimidin-5-yl)methyl]-4-methyl-1,3-thiazol-3-ium-5-yl]ethanol, 2,6-dichloropyridine-4-carboxylic acid, 3-methoxybenzo[d]isothiazole 1,1-dioxide, N-(3-chloro-4-methylphenyl)-4-methylthiadiazole-5-carboxamide, 3,4-dichloro-N-(2-cyanophenyl)-1,2-thiazole-5-carboxamide, 2-amino-3,5-dichlorobenzoic acid, 3-(furan-2-yl)-3-phenylpropan-1-amine, 4-amino-N-(5-methoxypyrimidin-2-yl)benzenesulfonamide, 4-amino-N-(6-methoxypyridazin-3-yl)benzenesulfonamide, N-(4-aminophenyl)sulfonylbenzamide, 4-amino-N-(6-chloropyridazin-3-yl)benzenesulfonamide, 4-amino-N-(5-methyl-1,2-oxazol-3-yl)benzenesulfonamide, 3-(butylamino)-4-phenoxy-5-sulfamoylbenzoic acid, 3-benzyl-1,1-dioxo-6-(trifluoromethyl)-3,4-dihydro-2H-126,2,4-benzothiadiazine-7-sulfonamide, 4-chloro-N-[(2R,6S)-2,6-dimethylpiperidin-1-yl]-3-sulfamoylbenzamide, (3R,4S,5R,6R)-4-[(3R,4S,5R,6R)-3,5-dihydroxy-6-(hydroxymethyl)-4-[(3R,4S,5S,6R)-3,4,5-trihydroxy-6-(hydroxymethyl) oxan-2-yl]oxyoxan-2-yl]oxy-6-(hydroxymethyl) oxane-2,3,5-triol, (3R,6S)-3-hydroxy-3-[(3-hydroxyphenyl)methyl]-1,4-dimethyl-6-[(4-nitro-1H-indol-3-yl)methyl]piperazine-2,5-dione, 2,4-dichloro-6-[(3-methoxyphenyl)iminomethyl]phenol, 1,2-benzothiazole 1,1-dioxide, 5-[(3-carboxy-4-hydroxyphenyl)-(3-carboxy-4-oxocyclohexa-2,5-dien-1-ylidene)methyl]-2-hydroxybenzoic acid, disodium 6-methyl-2-[4-[2-[4-(6-methyl-7-sulfonato-1,3-benzothiazol-2-yl)phenyl]iminohydrazinyl]phenyl]-1,3-benzothiazole-7-sulfonate, 8-[[4-methyl-3-[[3-[[3-[[2-methyl-5-[(4,6,8-trisulfonaphthalen-1-yl)carbamoyl]phenyl]carbamoyl]phenyl]carbamoylamino]benzoyl]amino]benzoyl]amino]nap hthalene-1,3,5-trisulfonic acid, 1-[4-Fluoro-3-(trifluoromethyl)phenyl]-3-(5-pyridin-4-yl-1,3,4-thiadiazol-2-yl) urea, 3-[18-(2-carboxyethyl)-7,12-diethyl-3,8,13,17,22-pentamethyl-23H-porphyrin-2-yl]propanoic acid, 1h-pyrrole-2,5-dione, 1-[(1-oxopropoxy)methyl]-N-propionyloxymethyl-maleimide, 3,4-dichloro-1-[3-(3,4-dichloro-2,5-dioxopyrrol-1-yl)-2,2-dimethylpropyl]pyrrole-2,5-dione, 5,7-dioxa-12-azoniapentacyclo[10.6.1.02,10.04,8.015,19]nonadeca-1 (18),2,4 (8),9,11,15 (19),16-heptaen-17-ol, 5,6-bis(((E)-benzylidene)amino)-2-thioxo-2,3-dihydropyrimidin-4 (1H)-one, (6Z)-3,7,11-trimethyldodeca-1,6,10-trien-3-ol, (2S,5R)-5-methyl-2-propan-2-ylcyclohexan-1-one, (1S,2S,5S,8R,9S, 10S, 11R, 15S, 18R)-9,10,15, 18-tetrahydroxy-12, 12-dimethyl-6-methylidene-17-oxapentacyclo[7.6.2.15,8.01,11.02,8]octadecan-7-one, (1R,2R,5R,9R,12R, 16R)-5-ethenyl-1,5,12-trimethyl-10-oxatetracyclo[7.6.1.02,7.012,16]hexadec-7-ene-11,13-dione, (1S,2R,5R, 12R, 18R)-5-ethenyl-13-hydroxy-5,12-dimethyl-10,14-dioxapentacyclo[11.2.2.11,9.02,7.012,18]octadec-7-en-11-one, [(1R,2R,4S,6S,9R, 10S, 13S, 16R)-2, 16-dihydroxy-5,5,9-trimethyl-14-methylidene-15-oxo-6-tetracyclo[11.2.1.01,10.04,9]hexadecanyl]acetate, (1S,4S,8R,9R,12S, 13S, 14S, 16S)-9, 14-dihydroxy-7,7-dimethyl-17-methylidene-3,10-dioxapentacyclo[14.2.1.01,13.04,12.08,12]nonadecane-2,18-dione, [(1S, 1′R,3'S,5R,6S, 7S,9S)-3′-acetyloxy-7-hydroxy-6′,6′-dimethyl-10-methylidene-2,11-dioxospiro[3-oxatricyclo[7.2.1.01,6]dodecane-5,2′-cyclohexane]-1′-yl]methyl acetate, (3E,6E)-3,7,11-trimethyldodeca-1,3,6,10-tetraene, 6,6-dimethyl-2-methylidenebicyclo[3.1.1]heptane, 2,2-dimethyl-3-methylidenebicyclo[2.2.1]heptane, 5-methyl-2-propan-2-ylphenol, (2E)-3,7-dimethylocta-2,6-dien-1-ol, (2E)-3,7-dimethylocta-2,6-dienal, 5-methyl-2-propan-2-ylcyclohexan-1-ol, benzoic acid, 2-aminophenoxazin-3-one, 2-amino-7-methoxyphenoxazin-3-one, (2S,3R)-2-(3,4-dihydroxyphenyl)-3,4-dihydro-2H-chromene-3,5,7-triol, and any combination thereof.

In some embodiments, a compound that modulates meiotic recombination is a compound that affects a component of and/or used in a meiotic recombination pathway in a plant. In some embodiments, a compound that modulates meiotic recombination is selected from the group consisting of: ethyl 4-[(1-hydroxy-2-phenylindol-3-yl)-pyridin-2-ylmethyl]piperazine-1-carboxylate, N-(5-tert-butyl-1H-pyrazol-3-yl)-2-[(3R)-3-propan-2-ylpiperazin-1-yl]-7H-pyrrolo[2,3-d]pyrimidin-4-amine, N-(6-aminohexyl)-5-chloro-1-naphthalenesulfonamide, 2-chloro-10-(3-dimethylaminopropyl) phenothiazine hydrochloride, Z-5-(4-hydroxybenzylidene)-2-imino-1,3-thiazolidin-4-one, 2-[(2R)-2-methylpyrrolidin-2-yl]-1H-benzimidazole-4-carboxamide, 5-[(E)-2-(4-hydroxyphenyl) ethenyl]benzene-1,3-diol, 3-(benzylsulfamoyl)-4-bromo-N-(4-bromophenyl)benzamide, 2-(4-Morpholinyl)-6-(1-thianthrenyl)-4H-Pyran-4-one, an azane, a dichloroplatinum (2+) compound, cisplatin, 2-(pyridin-3-ylmethylidene) indene-1,3-dione, [(1S,2S,4R)-4-[4-[[(1S)-2,3-dihydro-1H-inden-1-yl]amino]pyrrolo[2,3-d]pyrimidin-7-yl]-2-hydroxycyclopentyl]methyl sulfamate, 2-[2-(3,4-dimethoxyphenyl)ethylamino]-6-(3-fluorophenyl)-8H-pyrimido[4,5-d]pyrimidine-5,7-dione, 1-(4-((dimethylamino)methyl)phenyl)-8,9-dihydro-2,7,9a-triazabenzo[cd]azulen-6 (7H)-one, (2E,4E,6R)-7-[4-(dimethylamino)phenyl]-N-hydroxy-4,6-dimethyl-7-oxohepta-2,4-dienamide, 4-[(E)-2-(3,5-dimethoxyphenyl) ethenyl]phenol, and any combination thereof.

In some embodiments, a compound that modulates meiotic recombination is a compound that affects an epigenetic mark (e.g., DNA methylation, post-translational modification of histone tails, deposition of variant histones and/or the action of small RNAs) and/or an epigenetic modifier in a plant. In some embodiments, a compound that modulates meiotic recombination is selected from the group consisting of: 4-amino-N-(4,6-dimethylpyrimidin-2-yl)benzenesulfonamide, (2S)-2-amino-4-ethylsulfanylbutanoic acid, 4-amino-1-[(2R,3R,4S,5R)-3,4-dihydroxy-5-(hydroxymethyl)oxolan-2-yl]-1,3,5-triazin-2-one, sodium butanoate, 5,7-dihydroxy-3-(4-hydroxyphenyl) chromen-4-one, pyridine-2,4-dicarboxylic acid, (1R,2S)-2-phenylcyclopropan-1-amine, 4-amino-1-[(2R,4S,5R)-4-hydroxy-5-(hydroxymethyl)oxolan-2-yl]-1,3,5-triazin-2-one, 1-[(2R,3R,4S,5R)-3,4-dihydroxy-5-(hydroxymethyl)oxolan-2-yl]pyrimidin-2-one, 3-[4-[(1R,2S)-2-aminocyclopropyl]phenyl]phenol, methyl 3-[4-(4-carbamimidoylbenzoyl) piperazine-1-carbonyl]-5-[(4-carbamimidoylpiperazin-1-yl)methyl]benzoate, 2-[[[2-[2-(dimethylamino)ethyl-ethylamino]-2-oxoethyl]amino]methyl]pyridine-4-carboxamide, 2-(4-methylphenyl)-1,2-benzothiazol-3-one, 5-chloro-N-[(E)-[phenyl(pyridin-2-yl)methylidene]amino]pyridin-2-amine, methyl 2-benzamido-1-(3-phenylpropyl)benzimidazole-5-carboxylate, 2-[(2-hydroxynaphthalen-1-yl)methylideneamino]-N-(1-phenylethyl)benzamide, 3-(prop-2-enyldisulfanyl) prop-1-ene, N-(1-benzylpiperidin-4-yl)-6,7-dimethoxy-2-(4-methyl-1,4-diazepan-1-yl) quinazolin-4-amine, 5′-methoxy-6′-(3-pyrrolidin-1-ylpropoxy)spiro[cyclobutane-1,3′-indole]-2′-amine, 2-cyclohexyl-6-methoxy-N-(1-propan-2-ylpiperidin-4-yl)-7-(3-pyrrolidin-1-ylpropoxy) quinazolin-4-amine, 7-[3-(dimethylamino) propoxy]-6-methoxy-2-(4-methyl-1,4-diazepan-1-yl)-N-(1-methylpiperidin-4-yl) quinazolin-4-amine, 14-(hydroxymethyl)-3-[14-(hydroxymethyl)-18-methyl-13,17-dioxo-15,16-dithia-10,12,18-triazapentacyclo[12.2.2.01,12.03,11.04,9]octadeca-4,6,8-trien-3-yl]-18-methyl-15,16-dithia-10,12,18-triazapentacyclo[12.2.2.01,12.03,11.04,9]octadeca-4,6,8-triene-13,17-dione, and any combination thereof.

In some embodiments, a compound that modulates meiotic recombination may be a compound that mimics temperature shock in a plant. In some embodiments, the compound mimics heat shock (e.g., heat stress) in the plant and/or the compound mimics cold shock (e.g., cold stress) in the plant. In some embodiments, a compound that modulates meiotic recombination is selected from the group consisting of: 2,3-dichloro-1,4-naphthoquinone, 2,3-dichloro-5,8-dihydroxy-1,4-naphthoquinone, (1S,2R,4S)-1,7,7-trimethylbicyclo[2.2.1]heptan-2-ol, 2-isothiocyanatoethylbenzene, 4-isothiocyanatobutylbenzene, 3-isothiocyanatoprop-1-ene, isothiocyanatoethane, 1-isothiocyanatobutane, 3-isothiocyanato-2-methylprop-1-ene, [(4E,6Z,8S,9S,10E,12S, 13R, 14S, 16R)-13-hydroxy-8,14, 19-trimethoxy-4, 10, 12, 16-tetramethyl-3,20,22-trioxo-2-azabicyclo[16.3.1]docosa-1 (21),4,6,10, 18-pentaen-9-yl]carbamate, 4-[[(2R,3S,4R,5R)-5-[6-amino-8-[(3,4-dichlorophenyl)methylamino]purin-9-yl]-3,4-dihydroxyoxolan-2-yl]methoxymethyl]benzonitrile, 24-methyl-5,7,18,20-tetraoxa-24-azoniahexacyclo[11.11.0.02,10.04,8.014,22.017,21]tetracosa-1 (24),2,4 (8),9,11,13,15,17 (21),22-nonaene, (4S)-4-prop-1-en-2-ylcyclohexene-1-(4R)-1-methyl-4-prop-1-en-2-ylcyclohexene, (1R,4R)-1,7,7-carbaldehyde, trimethylbicyclo[2.2.1]heptan-2-one, 1,3,3-trimethyl-2-oxabicyclo[2.2.2]octane, 2,6,6-trimethylbicyclo[3.1.1]hept-2-ene, isothiocyanatobenzene, 2-isothiocyanatopropane, 1-isothiocyanato-2-methylpropane, 1-isothiocyanatohexane, 1-isothiocyanato-4-methylsulfinylbutane, 1-isothiocyanato-5-methylsulfinylpentane, 8-[(6-iodo-1,3-benzodioxol-5-yl)sulfanyl]-9-[3-(propan-2-ylamino) propyl]purin-6-amine, [4-[2-carbamoyl-5-[6,6-dimethyl-4-oxo-3-(trifluoromethyl)-5,7-dihydroindazol-1-yl]anilino]cyclohexyl]2-aminoacetate, 3-(2,4-dihydroxy-5-propan-2-ylphenyl)-4-(1-methylindol-5-yl)-1H-1,2,4-triazol-5-one, and any combination thereof. In some embodiments, a compound that modulates meiotic recombination is N-acetyl-5-methoxytryptamine.

In some embodiments, a compound that modulates meiotic recombination may be a compound that mimics pathogen stress in a plant. In some embodiments, a compound that modulates meiotic recombination is selected from the group consisting of: S-methyl 1,2,3-benzothiadiazole-7-carbothioate, 5-chloro-2-methylpyrazole-3-carboxylic acid, 3-prop-2-enoxy-1,2-benzothiazole 1,1-dioxide, 3-aminobutanoic acid, 1,1-dioxo-1,2-benzothiazol-3-one, 4-hydroxybenzohydrazide, 3-hydroxy-3-(2-oxopropyl)-1H-indol-2-one, 2-[3-[(4-amino-2-methylpyrimidin-5-yl)methyl]-4-methyl-1,3-thiazol-3-ium-5-yl]ethanol, 2,6-dichloropyridine-4-carboxylic acid, 3-methoxybenzo[d]isothiazole 1,1-dioxide, N-(3-chloro-4-methylphenyl)-4-methylthiadiazole-5-carboxamide, 3,4-dichloro-N-(2-cyanophenyl)-1,2-thiazole-5-carboxamide, 2-amino-3,5-dichlorobenzoic acid, 3-(furan-2-yl)-3-phenylpropan-1-amine, 4-amino-N-(5-methoxypyrimidin-2-yl)benzenesulfonamide, 4-amino-N-(6-methoxypyridazin-3-yl)benzenesulfonamide, N-(4-aminophenyl)sulfonylbenzamide, 4-amino-N-(6-chloropyridazin-3-yl)benzenesulfonamide, 4-amino-N-(5-methyl-1,2-oxazol-3-yl)benzenesulfonamide, 3-(butylamino)-4-phenoxy-5-sulfamoylbenzoic acid, 3-benzyl-1,1-dioxo-6-(trifluoromethyl)-3,4-dihydro-2H-126,2,4-benzothiadiazine-7-sulfonamide, 4-chloro-N-[(2R,6S)-2,6-dimethylpiperidin-1-yl]-3-sulfamoylbenzamide, (3R,4S,5R,6R)-4-[(3R,4S,5R,6R)-3,5-dihydroxy-6-(hydroxymethyl)-4-[(3R,4S,5S,6R)-3,4,5-trihydroxy-6-(hydroxymethyl) oxan-2-yl]oxyoxan-2-yl]oxy-6-(hydroxymethyl) oxane-2,3,5-triol, (3R,6S)-3-hydroxy-3-[(3-hydroxyphenyl)methyl]-1,4-dimethyl-6-[(4-nitro-1H-indol-3-yl)methyl]piperazine-2,5-dione, 2,4-dichloro-6-[(3-methoxyphenyl)iminomethyl]phenol, 1,2-benzothiazole 1,1-dioxide, and any combination thereof.

In some embodiments, a compound that modulates meiotic recombination may be a compound that affects helicase activity and/or formation in a plant, optionally wherein the compound modulates the activity of an enzyme (e.g., a helicase). In some embodiments, a compound that modulates meiotic recombination is selected from the group consisting of: 5-[(3-carboxy-4-hydroxyphenyl)-(3-carboxy-4-oxocyclohexa-2,5-dien-1-ylidene)methyl]-2-hydroxybenzoic acid, disodium 6-methyl-2-[4-[2-[4-(6-methyl-7-sulfonato-1,3-benzothiazol-2-yl)phenyl]iminohydrazinyl]phenyl]-1,3-benzothiazole-7-sulfonate, 8-[[4-methyl-3-[[3-[[3-[[2-methyl-5-[(4,6,8-trisulfonaphthalen-1-yl)carbamoyl]phenyl]carbamoyl]phenyl]carbamoylamino]benzoyl]amino]benzoyl]amino]nap hthalene-1,3,5-trisulfonic acid, 1-[4-Fluoro-3-(trifluoromethyl)phenyl]-3-(5-pyridin-4-yl-1,3,4-thiadiazol-2-yl) urea, 3-[18-(2-carboxyethyl)-7,12-diethyl-3,8,13, 17,22-pentamethyl-23H-porphyrin-2-yl]propanoic acid, 1h-pyrrole-2,5-dione, 1-[(1-oxopropoxy)methyl]-N-propionyloxymethyl-maleimide, 3,4-dichloro-1-[3-(3,4-dichloro-2,5-dioxopyrrol-1-yl)-2,2-dimethylpropyl]pyrrole-2,5-dione, and any combination thereof.

In some embodiments, a compound that modulates meiotic recombination may be a compound that inhibits non-homologous end joining (NHEJ) in a plant. Meiotic recombination is initiated by the creation of double strand breaks (DSBs) in DNA. Several cellular pathways are capable of repairing DSBs, but homologous recombination (HR) is the primary pathway that is useful to breeders. In some embodiments, a method and/or compound that modulates meiotic recombination may inhibit a pathway that competes with HR, which may force the cell to rely more heavily on HR and may result in an increase in the frequency of meiotic recombination events. In some embodiments, a compound that modulates meiotic recombination is selected from the group consisting of: 5,7-dioxa-12-azoniapentacyclo[10.6.1.02,10.04,8.015,19]nonadeca-1 (18),2,4 (8),9,11,15 (19), 16-heptaen-17-ol, and any combination thereof. In some embodiments, a compound that modulates meiotic recombination is 5,6-bis(((E)-benzylidene)amino)-2-thioxo-2,3-dihydropyrimidin-4 (1H)-one.

In some embodiments, a compound that modulates meiotic recombination may be an allelopathic compound. In some embodiments, a compound that modulates meiotic recombination is selected from the group consisting of: (6Z)-3,7,11-trimethyldodeca-1,6,10-trien-3-ol, (2S,5R)-5-methyl-2-propan-2-ylcyclohexan-1-one, (1S,2S,5S,8R,9S,10S,11R, 15S, 18R)-9,10, 15, 18-tetrahydroxy-12,12-dimethyl-6-methylidene-17-oxapentacyclo[7.6.2.15,8.01, 11.02,8]octadecan-7-one, (1R,2R,5R,9R,12R,16R)-5-ethenyl-1,5,12-trimethyl-10-oxatetracyclo[7.6.1.02,7.012,16]hexadec-7-ene-11,13-dione, (1S,2R,5R, 12R, 18R)-5-ethenyl-13-hydroxy-5,12-dimethyl-10,14-dioxapentacyclo[11.2.2.11,9.02,7.012, 18]octadec-7-en-11-one, [(1R,2R,4S,6S,9R, 10S, 13S, 16R)-2, 16-dihydroxy-5,5,9-trimethyl-14-methylidene-15-oxo-6-tetracyclo[11.2.1.01,10.04,9]hexadecanyl]acetate, (1S,4S,8R,9R,12S, 13S, 14S, 16S)-9,14-dihydroxy-7,7-dimethyl-17-methylidene-3,10-dioxapentacyclo[14.2.1.01,13.04,12.08,12]nonadecane-2,18-dione, [(1S, 1′R,3'S,5R, 6S, 7S,9S)-3′-acetyloxy-7-hydroxy-6′,6′-dimethyl-10-methylidene-2,11-dioxospiro[3-oxatricyclo[7.2.1.01,6]dodecane-5,2′-cyclohexane]-1′-yl]methyl acetate, (3E,6E)-3,7,11-trimethyldodeca-1,3,6,10-tetraene, 6,6-dimethyl-2-methylidenebicyclo[3.1.1]heptane, 2,2-dimethyl-3-methylidenebicyclo[2.2.1]heptane, 5-methyl-2-propan-2-ylphenol, (2E)-3,7-dimethylocta-2,6-dien-1-ol, (2E)-3,7-dimethylocta-2,6-dienal, 5-methyl-2-propan-2-ylcyclohexan-1-ol, benzoic acid, 2-aminophenoxazin-3-one, 2-amino-7-methoxyphenoxazin-3-one, (2S,3R)-2-(3,4-dihydroxyphenyl)-3,4-dihydro-2H-chromene-3,5,7-triol, and any combination thereof.

In some embodiments, a method of the present invention comprises applying a compound that modulates meiotic recombination to a plant, wherein the compound is a compound of Table 1. In some embodiments, the method comprises applying two or more (e.g., 2, 3, 4, 5, or more) compounds of Table 1 to the plant. In some embodiments, the target and/or function of the compound modulates meiotic recombination is as provided in Table 1.

TABLE 1
Exemplary target and/or exemplary function for a compound that
modulates meiotic recombination of the present invention.

| Exemplary | |
|---|---|
| target | |
| and/or | |
| exemplary | |
| function | Compound that modulates meiotic recombination |
| FANCM | ethyl 4-[(1-hydroxy-2-phenylindol-3-yl)-pyridin-2-ylmethyl]piperazine-1- |
| | carboxylate |
| FIGL1 | N-(5-tert-butyl-1H-pyrazol-3-y1)-2-[(3R)-3-propan-2-ylpiperazin-1-y1]-7H- |
| | pyrrolo[2,3-d]pyrimidin-4-amine |
| Ku70/80 | N-(6-aminohexyl)-5-chloro-1-naphthalenesulfonamide |
| Ku70/80 | 2-chloro-10-(3-dimethylaminopropyl)phenothiazine hydrochloride |
| MRE11 | Z-5-(4-hydroxybenzylidene)-2-imino-1,3-thiazolidin-4-one |
| PARP1 | 2-[(2R)-2-methylpyrrolidin-2-y1]-1H-benzimidazole-4-carboxamide |
| RAD51 | 5-[(E)-2-(4-hydroxyphenyl)ethenyl]benzene-1,3-diol |
| RAD51 | 3-(benzylsulfamoyl)-4-bromo-N-(4-bromophenyl)benzamide |
| ATM | 2-(4-Morpholiny1)-6-(1-thianthrenyl)-4H-Pyran-4-one, 2-(Morpholin-4-yl)-6- |
| | (thianthren-1-y1)-4H-pyran-4-one |
| [general] | azane; dichloroplatinum(2+) |
| H2AX | 2-(pyridin-3-ylmethylidene)indene-1,3-dione |
| COM1/SAE2 | [(1S,2S,4R)-4-[4-[[(1S)-2,3-dihydro-1H-inden-1-yl]amino]pyrrolo[2,3- |
| | d]pyrimidin-7-yl]-2-hydroxycyclopentyl]methyl sulfamate |
| Ku70/80 | 2-[2-(3,4-dimethoxyphenyl)ethylamino]-6-(3-fluorophenyl)-8H-pyrimido[4,5- |
| | d]pyrimidine-5,7-dione |
| PARP1 | 1-(4-((dimethylamino)methyl)phenyl)-8,9-dihydro-2,7,9a- |
| | triazabenzo[cd]azulen-6(7H)-one |
| RAD51 | (2E,4E,6R)-7-[4-(dimethylamino)phenyl]-N-hydroxy-4,6-dimethyl-7-oxohepta- |
| | 2,4-dienamide |
| RAD51 | 4-[(E)-2-(3,5-dimethoxyphenyl)ethenyl]phenol |
| DNA | 4-amino-N-(4,6-dimethylpyrimidin-2-yl)benzenesulfonamide |
| methylation | |
| DNA | 2S)-2-amino-4-ethylsulfanylbutanoic acid |
| methylation | |
| DNA | 4-amino-1-[(2R,3R,4S,5R)-3,4-dihydroxy-5-(hydroxymethyl)oxolan-2-yl]- |
| methylation | 1,3,5-triazin-2-one |
| Histone | Sodium butanoate |
| deacetylase | |
| inhibitor | |
| Non-CG | 5,7-dihydroxy-3-(4-hydroxyphenyl)chromen-4-one |
| DNA | |
| methylation, | |
| CMT3 | |
| H3K4me3 | pyridine-2,4-dicarboxylic acid |
| demethylase | |
| inhibitor, | |
| LSD1 | |
| H3K4me3 | (1R,2S)-2-phenylcyclopropan-1-amine |
| demethylase | |
| inhibitor, | |
| LSD1 | |
| DNA | 4-amino-1-[(2R,4S,5R)-4-hydroxy-5-(hydroxymethyl)oxolan-2-yl]-1,3,5- |
| methylation | triazin-2-one |
| DNA | 1-[(2R,3R,4S,5R)-3,4-dihydroxy-5-(hydroxymethyl)oxolan-2-yl]pyrimidin-2- |
| methylation | one |
| H3K4me3 | 3-[4-[(1R,2S)-2-aminocyclopropyl]phenyl]phenol |
| demethylase | |
| inhibitor, | |
| LSD1 | |
| H3K4me3 | methyl 3-[4-(4-carbamimidoylbenzoyl)piperazine-1-carbonyl]-5-[(4- |
| demethylase | carbamimidoylpiperazin-1-yl)methyl]benzoate |
| inhibitor, | |
| LSD1 | |
| H3K4me3 | 2-[[[2-[2-(dimethylamino)ethyl-ethylamino]-2- |
| demethylase | oxoethyl]amino]methyl]pyridine-4-carboxamide |
| inhibitor, | |
| LSD1 | |
| H3K4me3 | 2-(4-methylphenyl)-1,2-benzothiazol-3-one |
| demethylase | |
| inhibitor, | |
| LSD1 | |
| H3K4me3 | 5-chloro-N-[(E)-[phenyl(pyridin-2-yl)methylidene]amino]pyridin-2-amine |
| demethylase | |
| inhibitor, | |
| LSD1 | |
| H3K4me3 | methyl 2-benzamido-1-(3-phenylpropyl)benzimidazole-5-carboxylate |
| demethylase | |
| inhibitor, | |
| LSD1 | |
| Histone | 2-[(2-hydroxynaphthalen-1-yl)methylideneamino]-N-(1-phenylethyl)benzamide |
| deacetylase | |
| inhibitor | |
| Histone | 3-(prop-2-enyldisulfanyl)prop-1-ene |
| deacetylase | |
| inhibitor | |
| Inhibit | N-(1-benzylpiperidin-4-yl)-6,7-dimethoxy-2-(4-methyl-1,4-diazepan-1- |
| H3K9 | yl)quinazolin-4-amine |
| methylation, | |
| SDG21 | |
| Inhibit | 5′-methoxy-6′-(3-pyrrolidin-1-ylpropoxy)spiro[cyclobutane-1,3′-indole]-2′- |
| H3K9 | amine |
| methylation, | |
| SDG21 | |
| Inhibit | 2-cyclohexyl-6-methoxy-N-(1-propan-2-ylpiperidin-4-yl)-7-(3-pyrrolidin-1- |
| H3K9 | ylpropoxy)quinazolin-4-amine |
| methylation, | |
| SDG21 | |
| Inhibit | 7-[3-(dimethylamino)propoxy]-6-methoxy-2-(4-methyl-1,4-diazepan-1-yl)-N- |
| H3K9 | (1-methylpiperidin-4-yl)quinazolin-4-amine |
| methylation, | |
| SDG21 | |
| Inhibit | 14-(hydroxymethyl)-3-[14-(hydroxymethyl)-18-methyl-13,17-dioxo-15,16- |
| H3K9 | dithia-10,12,18-triazapentacyclo[12.2.2.01,12.03,11.04,9]octadeca-4,6,8-trien- |
| methylation, | 3-y1]-18-methyl-15,16-dithia-10,12,18- |
| SUVH4/5/6 | triazapentacyclo[12.2.2.01,12.03,11.04,9]octadeca-4,6,8-triene-13,17-dione |
| HSP90 | 2,3-dichloro-1,4-naphthoquinone |
| HSP90 | 2,3-dichloro-5,8-dihydroxy-1,4-naphthoquinone |
| HSP90 | (1S,2R,4S)-1,7,7-trimethylbicyclo[2.2.1]heptan-2-ol |
| HSP90 | 2-isothiocyanatoethylbenzene |
| HSP90 | 4-isothiocyanatobutylbenzene |
| HSP90 | 3-isothiocyanatoprop-1-ene |
| HSP90 | isothiocyanatoethane |
| HSP90 | 1-isothiocyanatobutane |
| HSP90 | 3-isothiocyanato-2-methylprop-1-ene |
| HSP90 | [(4E,6Z,8S,9S,10E,12S,13R,14S,16R)-13-hydroxy-8,14,19-trimethoxy- |
| | 4,10,12,16-tetramethyl-3,20,22-trioxo-2-azabicyclo[16.3.1]docosa- |
| | 1(21),4,6,10,18-pentaen-9-y1] carbamate |
| HSP90 | 4-[[(2R,3S,4R,5R)-5-[6-amino-8-[(3,4-dichlorophenyl)methylamino]purin-9- |
| | yl]-3,4-dihydroxyoxolan-2-yl]methoxymethyl]benzonitrile |
| HSP90 | 24-methyl-5,7,18,20-tetraoxa-24- |
| | azoniahexacyclo[11.11.0.02,10.04,8.014,22.017,21]tetracosa- |
| | 1(24),2,4(8),9,11,13,15,17(21),22-nonaene |
| HSP90 | (4S)-4-prop-1-en-2-ylcyclohexene-1-carbaldehyde |
| HSP90 | (4R)-1-methyl-4-prop-1-en-2-ylcyclohexene |
| HSP90 | (1R,4R)-1,7,7-trimethylbicyclo[2.2.1]heptan-2-one |
| HSP90 | 1,3,3-trimethyl-2-oxabicyclo[2.2.2]octane |
| HSP90 | 2,6,6-trimethylbicyclo[3.1.1]hept-2-ene |
| HSP90 | isothiocyanatobenzene |
| HSP90 | 2-isothiocyanatopropane |
| HSP90 | 1-isothiocyanato-2-methylpropane |
| HSP90 | 1-isothiocyanatohexane |
| HSP90 | 1-isothiocyanato-4-methylsulfinylbutane |
| HSP90 | 1-isothiocyanato-5-methylsulfinylpentane |
| HSP90 | 8-[(6-iodo-1,3-benzodioxol-5-yl)sulfanyl]-9-[3-(propan-2- |
| | ylamino)propyl]purin-6-amine |
| HSP90 | [4-[2-carbamoyl-5-[6,6-dimethyl-4-oxo-3-(trifluoromethyl)-5,7- |
| | dihydroindazol-1-yl]anilino]cyclohexyl] 2-aminoacetate |
| HSP90 | 3-(2,4-dihydroxy-5-propan-2-ylphenyl)-4-(1-methylindol-5-yl)-1H-1,2,4- |
| | triazol-5-one |
| mimic cold | N-acetyl-5-methoxytryptamine |
| stress | |
| mimic | S-methyl 1,2,3-benzothiadiazole-7-carbothioate |
| pathogen | |
| stress | |
| mimic | 5-chloro-2-methylpyrazole-3-carboxylic acid |
| pathogen | |
| stress | |
| mimic | 3-prop-2-enoxy-1,2-benzothiazole 1,1-dioxide |
| pathogen | |
| stress | |
| mimic | 3-aminobutanoic acid |
| pathogen | |
| stress | |
| mimic | 1,1-dioxo-1,2-benzothiazol-3-one |
| pathogen | |
| stress | |
| pathogen | 4-hydroxybenzohydrazide |
| stress | |
| mimic | 3-hydroxy-3-(2-oxopropyl)-1H-indol-2-one |
| pathogen | |
| stress | |
| mimic | 2-[3-[(4-amino-2-methylpyrimidin-5-yl)methyl]-4-methyl-1,3-thiazol-3-ium-5- |
| pathogen | yllethanol |
| stress | |
| mimic | 2,6-dichloropyridine-4-carboxylic acid |
| pathogen | |
| stress | |
| mimic | 3-Methoxybenzo[d]isothiazole 1,1-dioxide |
| pathogen | |
| stress | |
| mimic | N-(3-chloro-4-methylphenyl)-4-methylthiadiazole-5-carboxamide |
| pathogen | |
| stress | |
| mimic | 3,4-dichloro-N-(2-cyanophenyl)-1,2-thiazole-5-carboxamide |
| pathogen | |
| stress | |
| mimic | 2-Amino-3,5-dichlorobenzoic acid |
| pathogen | |
| stress | |
| mimic | 3-(furan-2-yl)-3-phenylpropan-1-amine |
| pathogen | |
| stress | |
| mimic | 4-amino-N-(5-methoxypyrimidin-2-yl)benzenesulfonamide |
| pathogen | |
| stress | |
| mimic | 4-amino-N-(6-methoxypyridazin-3-yl)benzenesulfonamide |
| pathogen | |
| stress | |
| mimic | N-(4-aminophenyl)sulfonylbenzamide |
| pathogen | |
| stress | |
| mimic | 4-amino-N-(6-chloropyridazin-3-yl)benzenesulfonamide |
| pathogen | |
| stress | |
| mimic | 4-amino-N-(5-methyl-1,2-oxazol-3-yl)benzenesulfonamide |
| pathogen | |
| stress | |
| mimic | 3-(butylamino)-4-phenoxy-5-sulfamoylbenzoic acid |
| pathogen | |
| stress | |
| mimic | 3-benzyl-1,1-dioxo-6-(trifluoromethyl)-3,4-dihydro-2H-1λ6,2,4- |
| pathogen | benzothiadiazine-7-sulfonamide |
| stress | |
| mimic | 4-chloro-N-[(2R,6S)-2,6-dimethylpiperidin-1-yl]-3-sulfamoylbenzamide |
| pathogen | |
| stress | |
| mimic | (3R,4S,5R,6R)-4-[(3R,4S,5R,6R)-3,5-dihydroxy-6-(hydroxymethyl)-4- |
| pathogen | [(3R,4S,5S,6R)-3,4,5-trihydroxy-6-(hydroxymethyl)oxan-2-yl]oxyoxan-2- |
| stress | yl]oxy-6-(hydroxymethyl)oxane-2,3,5-triol |
| mimic | (3R,6S)-3-hydroxy-3-[(3-hydroxyphenyl)methyl]-1,4-dimethyl-6-[(4-nitro-1H- |
| pathogen | indol-3-yl)methyl]piperazine-2,5-dione |
| stress | |
| mimic | 2,4-dichloro-6-[(3-methoxyphenyl)iminomethyl]phenol |
| pathogen | |
| stress | |
| mimic | 1,2-benzothiazole 1,1-dioxide |
| pathogen | |
| stress | |
| RECQ4A | 5-[(3-carboxy-4-hydroxyphenyl)-(3-carboxy-4-oxocyclohexa-2,5-dien-1- |
| (BLM) | ylidene)methyl]-2-hydroxybenzoic acid |
| RECQ4A | disodium; 6-methyl-2-[4-[2-[4-(6-methyl-7-sulfonato-1,3-benzothiazol-2- |
| (BLM) | y1)phenyl]iminohydrazinyl]phenyl]-1,3-benzothiazole-7-sulfonate |
| RECQ4A | 8-[[4-methyl-3-[3-[[3-[[2-methyl-5-[(4,6,8-trisulfonaphthalen-1- |
| (BLM) | yl)carbamoyl]phenyl]carbamoyl]phenyl]carbamoylamino]benzoyl]amino]ben- |
| | zoyl]amino]naphthalene-1,3,5-trisulfonic acid |
| RECQ4A | 1-[4-Fluoro-3-(trifluoromethyl)phenyl]-3-(5-pyridin-4-yl-1,3,4-thiadiazol-2- |
| (BLM) | yl)urea |
| RECQ4A | 3-[18-(2-carboxyethyl)-7,12-diethyl-3,8,13,17,22-pentamethyl-23H-porphyrin- |
| (BLM) | 2-yl]propanoic acid |
| RECQ4A | 1h-pyrrole-2,5-dione, 1-[(1-oxopropoxy)methyl]-N-propionyloxymethyl- |
| (BLM) | maleimide |
| RECQ4A | 3,4-dichloro-1-[3-(3,4-dichloro-2,5-dioxopyrrol-1-yl)-2,2- |
| (BLM) | dimethylpropyl]pyrrole-2,5-dione |
| TOP3A | 5,7-dioxa-12-azoniapentacyclo[10.6.1.02,10.04,8.015,19]nonadeca- |
| | 1(18),2,4(8),9,11,15(19),16-heptaen-17-ol |
| DNA ligase | 5,6-bis(((E)-benzylidene)amino)-2-thioxo-2,3-dihydropyrimidin-4(1H)-one |
| IV | |
| allelopathic | (6Z)-3,7,11-trimethyldodeca-1,6,10-trien-3-ol |
| allelopathic | (2S,5R)-5-methyl-2-propan-2-ylcyclohexan-1-one |
| allelopathic | (1S,2S,5S,8R,9S,10S,11R,15S,18R)-9,10,15,18-tetrahydroxy-12,12-dimethyl- |
| | 6-methylidene-17-oxapentacyclo[7.6.2.15,8.01,11.02,8]octadecan-7-one |
| allelopathic | (1R,2R,5R,9R,12R,16R)-5-ethenyl-1,5,12-trimethyl-10- |
| | oxatetracyclo[7.6.1.02,7.012,16]hexadec-7-ene-11,13-dione |
| allelopathic | (1S,2R,5R,12R,18R)-5-ethenyl-13-hydroxy-5,12-dimethyl-10,14- |
| | dioxapentacyclo[11.2.2.11,9.02,7.012,18]octadec-7-en-11-one |
| allelopathic | [(1R,2R,4S,6S,9R,10S,13S,16R)-2,16-dihydroxy-5,5,9-trimethyl-14- |
| | methylidene-15-oxo-6-tetracyclo[11.2.1.01,10.04,9]hexadecanyl] acetate |
| allelopathic | (1S,4S,8R,9R,12S,13S,14S,16S)-9,14-dihydroxy-7,7-dimethyl-17-methylidene- |
| | 3,10-dioxapentacyclo[14.2.1.01,13.04,12.08,12]nonadecane-2,18-dione |
| allelopathic | [(1S,1′R,3′S,5R,6S,7S,9S)-3′-acetyloxy-7-hydroxy-6′,6′-dimethyl-10- |
| | methylidene-2,11-dioxospiro[3-oxatricyclo[7.2.1.01,6]dodecane-5,2′- |
| | cyclohexane]-l′-yl]methyl acetate |
| allelopathic | (3E,6E)-3,7,11-trimethyldodeca-1,3,6,10-tetraene |
| allelopathic | 6,6-dimethyl-2-methylidenebicyclo[3.1.1]heptane |
| allelopathic | 2,2-dimethyl-3-methylidenebicyclo[2.2.1]heptane |
| allelopathic | 5-methyl-2-propan-2-ylphenol |
| allelopathic | (2E)-3,7-dimethylocta-2,6-dien-1-ol |
| allelopathic | (2E)-3,7-dimethylocta-2,6-dienal |
| allelopathic | 5-methyl-2-propan-2-ylcyclohexan-1-ol |
| allelopathic | benzoic acid |
| allelopathic | 2-aminophenoxazin-3-one |
| allelopathic | 2-amino-7-methoxyphenoxazin-3-one |
| allelopathic | (2S,3R)-2-(3,4-dihydroxyphenyl)-3,4-dihydro-2H-chromene-3,5,7-triol |

A compound that modulates meiotic recombination may be present in a composition (e.g., an aqueous composition). The compound may be present in the composition in an amount of about 0.001, 0.01, 0.05, 0.1, 0.5, 1, 1.5, 2, 2.5, 3, 4, or 5 mM to about 6, 7, 8, 9, 10, 11, 12, 13,14, 15, 16, 17, 18, 19, 20, 25, 30, 35, or 40 mM. In some embodiments, the compound may be present in the composition in an amount of about 0.001, 0.01, 0.05, 0.1, 0.5, 1, 1.5, 2, 2.5, 3, 4, 5, 6, 7, 8, 9, 10, 11, 12, 13, 14, 15, 16, 17, 18, 19, 20, 25, 30, 35, or 40 mM. In some embodiments, the composition comprises water and/or dimethylsulfoxide (DMSO). DMSO may be present in a composition in an amount of about 0.001%, 0.01%, 0.1%, 0.5%, 1%, or 2% to about 5%, 10%, 15%, or 20% by weight and/or by v/v of the composition. In some embodiments, DMSO may be present in a composition of the present invention in an amount of about 0.001% 0.01%, 0.1%, 0.5%, 1%, 2%, 5%, 10%, 15%, or 20% by weight and/or by v/v of the composition. A composition comprising a compound that modulates meiotic recombination may also include a surfactant such as, but not limited to, Tween-20 and/or Silwet, optionally at a concentration of about 0.001%, 0.01%, 0.1%, 0.025% to about 0.25% or 0.5% v/v of the composition. In some embodiments, a composition of the present invention comprises a surfactant such as, but not limited to, Tween-20 and/or Silwet, at a concentration of about 0.001%, 0.01%, 0.1%, 0.025%, 0.25% or 0.5% v/v of the composition. In some embodiments, a composition of the present invention comprises water, a compound that modulates meiotic recombination optionally in an amount of about 0.001, 0.01, 0.05, 0.1, 0.5, 1, 1.5, 2, 2.5, 3, 4, or 5 mM to about 6, 7, 8, 9, 10, 11, 12, 13, 14, 15, 16, 17, 18, 19, 20, 25, 30, 35, or 40 mM, DMSO in an amount of 10% by weight and/or by v/v of the composition, and a surfactant such as, but not limited to, Tween-20 and/or Silwet, at a concentration of about 0.01% v/v of the composition. In some embodiments, inclusion of DMSO and/or a surfactant may enhance delivery of the compound that modulates meiotic recombination to the plant.

In some embodiments, a method of the present invention improves linkage drag in a plant and/or its progeny such as by reducing linkage drag. During plant breeding desirable traits controlled by versions of genes (alleles) at genetic loci are selected for inclusion in elite commercial lines. Desirable trait loci can be located near (genetically linked) loci with alleles that impart undesirable traits. As a result, selecting the desirable trait, can “drag” the undesirable trait along with the desirable trait during breeding cycles. Separation of desirable traits from undesirable traits occurs when the DNA between the loci recombines (also known as a genetic exchange, or crossing-over) during meiosis. However, several factors limit the ability of crossovers to disrupt linkage drag. The number of crossovers a genome experiences in each meiosis is limited. In addition, the probability that a crossover occurs between two loci in inversely proportional to their distance from one another. To overcome these limitations breeders will use costly, time-intensive, labor-intensive and space-intensive strategies to screen though large populations to find the desired combination of alleles (genotypes). In some embodiments, by applying to a plant a compound of the present invention that increases meiotic recombination in the plant, linkage drag may be reduced.

In some embodiments, a method of the present invention increases recombination in a cold region (e.g., dead zone) of a plant genome. Crossovers are not distributed evenly across the genome during meiosis. Instead, there are crossover hotspots, crossover coldspots and regions that rarely if ever experience crossovers. Hotspots and coldspots (e.g., cold regions) are defined as regions of the genome that experience a statistically higher or lower, respectively, frequency of crossovers when compared to the genomic average for a given genotype (species, line, accession) in a given set of growth conditions. Alleles at loci in recombination cold/dead regions experience significantly fewer recombination events compared to the genomic average and as a result fewer new combinations of alleles (genotypes) are generated during meiosis. Plant breeders view these regions as a potentially rich source for “hidden” genetic variation-hidden in the sense that if recombination could be induced in these regions, novel genotypes with desirable phenotypes could be created. In some embodiments, by applying to a plant a compound of the present invention that increases meiotic recombination in the plant, an increase in recombination in a cold region may be achieved and/or hidden variation may be revealed.

In some embodiments, a method of the present invention eliminates the need for back crossing or decreases the amount of time for back crossing for a plant and/or its progeny. A method of the present invention may be devoid of a back crossing step. In some embodiments, a method of the present invention may reduce the number of backcross generations in a plant breeding method. During plant breeding experimental or wild accessions can be crossed with elite commercial lines to transfer desirable traits from the former into the latter via a process called introgression. Progeny from these crosses are then backcrossed to the elite parent to restore the elite parental genotype, reduce the experimental/wild genotype, and maintain the desirable trait. The efficiency of maximizing restoration of the elite genotype, and minimizing the experimental/wild genotype while maintaining the desirable trait is dependent on the frequency of meiotic recombination. To overcome limited meiotic recombination, plant breeders typically use several backcross generations which is costly, time-intensive, labor-intensive and space-intensive. In some embodiments, by applying to a plant a compound of the present invention that increases meiotic recombination in plants, the number of backcrossing generations could be reduced or eliminated in a plant breeding method.

In some embodiments, the plant in a method of the present invention is a crop plant (e.g., corn, tomato, rice, soybean, wheat, oilseed plant, etc.). In some embodiments, the plant in a method of the present invention is a monocot. In some embodiments, the plant in a method of the present invention is a eudicot. Non-limiting examples of plants useful with the present invention include turf grasses (e.g., bluegrass, bentgrass, ryegrass, fescue), feather reed grass, tufted hair grass, miscanthus, arundo, switchgrass, vegetable crops, including artichokes, kohlrabi, arugula, leeks, asparagus, lettuce (e.g., head, leaf, romaine), malanga, melons (e.g., muskmelon, watermelon, crenshaw, honeydew, cantaloupe), cole crops (e.g., brussels sprouts, cabbage, cauliflower, broccoli, collards, kale, Chinese cabbage, bok choy), cardoni, carrots, napa, okra, onions, celery, parsley, chick peas, parsnips, chicory, peppers, potatoes, cucurbits (e.g., marrow, cucumber, zucchini, squash, pumpkin, honeydew melon, watermelon, cantaloupe), radishes, dry bulb onions, rutabaga, eggplant, salsify, escarole, shallots, endive, garlic, spinach, green onions, squash, greens, beet (sugar beet and fodder beet), sweet potatoes, chard, horseradish, tomatoes, turnips, and spices; a fruit crop such as apples, apricots, cherries, nectarines, peaches, pears, plums, prunes, cherry, quince, fig, nuts (e.g., chestnuts, pecans, pistachios, hazelnuts, pistachios, peanuts, walnuts, macadamia nuts, almonds, and the like), citrus (e.g., clementine, kumquat, orange, grapefruit, tangerine, mandarin, lemon, lime, and the like), blueberries, black raspberries, boysenberries, cranberries, currants, gooseberries, loganberries, raspberries, strawberries, blackberries, grapes (wine and table), avocados, bananas, kiwi, persimmons, pomegranate, pineapple, tropical fruits, pomes, melon, mango, papaya, and lychee, a field crop plant such as clover, alfalfa, timothy, evening primrose, meadow foam, corn/maize (field, sweet, popcorn), hops, jojoba, buckwheat, safflower, quinoa, wheat, rice, barley, rye, millet, sorghum, oats, triticale, sorghum, tobacco, kapok, a leguminous plant (beans (e.g., green and dried), lentils, peas, soybeans), an oil plant (rape, canola, mustard, poppy, olive, sunflower, coconut, castor oil plant, cocoa bean, groundnut, oil palm), duckweed, Arabidopsis, a fiber plant (cotton, flax, hemp, jute), Cannabis (e.g., Cannabis sativa,Cannabis indica, and Cannabis ruderalis), lauraceae (cinnamon, camphor), or a plant such as coffee, sugar cane, tea, and natural rubber plants; and/or a bedding plant such as a flowering plant, a cactus, a succulent and/or an ornamental plant (e.g., roses, tulips, violets), as well as trees such as forest trees (broad-leaved trees and evergreens, such as conifers; e.g., elm, ash, oak, maple, fir, spruce, cedar, pine, birch, cypress, eucalyptus, willow), as well as shrubs and other nursery stock.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

An active ingredient (AI) (e.g., a compound that modulates meiotic recombination in a plant) will be applied to Arabidopsis plants using foliar spray, soil drench, or liquid media. Adjuvants such as DMSO, Tween-20, and/or Silwet may be used to enhance delivery of the AI, optionally by providing a liquid composition including the AI and water, DMSO, Tween-20, and/or Silwet.

Crossovers will be measured in pollen tetrads from replicate treated plants and compared to untreated or mock treated controls. Scoring as few as about 300 tetrads per treated plant or control is sufficient to detect modified (e.g., enhanced) crossover frequencies. Initially, each AI will be tested at a range of concentrations, for example 100 μM, 1 mM and 10 mM concentrations. If phytotoxicity for an AI is observed, then lower concentrations will be tested. For some AIs, a finer resolution of concentrations will be tested to develop a response curve.

An AI that increases or decreases meiotic recombination in an Arabidopsis plant will be validated by applying the AI to tomato plants and scoring the respective meiotic crossover frequencies. A visual fluorescent pollen transgene assay (e.g., a tomato-based version of the FTL system as described in Francis et al., Proc Natl Acad Sci USA. 2007; 104 (10): 3913-3918, Modliszewski et al., PLOS Genet, 2018, 14 (5): e1007384, and/or Berchowitz, L. and Copenhaver, G. Nature Protocols, 2008; 3 (1), 41-50) will be used to measure meiotic crossover (CO) frequency. Post-treatment pollen viability will also be assayed using a modified Alexander's staining procedure.

Success will be defined as identification of one or more AIs that statistically increase or decrease meiotic CO frequency in treated plants compared to untreated controls at a significance level of p≤0.05. An increase in CO frequency by 3X or more is viewed as useful of breeding purposes.

### Example 2

Meiotic recombination was stimulated in rice by external application of phenethyl isothiocyanate (IUPAC name: 2-isothiocyanatoethylbenzene). First, two parental rice lines, “Oryza sativa subspecies japonica variety Kitaake” (“Kitaake” hereafter) and “Oryza sativa subspecies japonica variety Nipponbare” (“Nipponbare” hereafter) were manually crossed to produce F1 seeds. F1 seeds were germinated and grown to produce F1 plants. PCR was used to genotype the F1 plants and confirm their heterozygosity. When F1 plants produced panicles (inflorescences) they were treated by spraying the whole plant, until saturation, with 7.5 mM phenethyl isothiocyanate in an aqueous solution containing 10% DMSO and 0.01% Silwet. The phenethyl isothiocyanate was first dissolved in 100% DMSO and then diluted to its final concentration with 0.0.1% Silwet in water. Control plants were mock-treated with 10% DMSO and 0.01% Silwet. Treated plants and controls were grown for 5-7 days and pollen was harvested. High molecular weight DNA was isolated from the pollen from treated plants, control plants, as well as from the vegetative tissue of parental plants. Isolated DNA was sequenced using an Oxford Nanopore Technologies sequencing platform with the ligation-based SQK-LSK109 sequencing kit for singleplex samples and the 9.4.1 flow cell. The “Super high accuracy—plant” option was used for base-calling. Single nucleotide polymorphisms (SNPs) were identified by comparing the Kitaake and Nipponbare parental sequences. Reads that spanned at least two parental SNPs were selected and binned into those that contained SNPs of only the Kitaake type (non-recombinant), only the Nipponbare type (non-recombinant) or both Kitaake and Nipponbare type (recombinant). The frequency of recombinant reads from treated and untreated plants were compared to detect an increase in meiotic recombination resulting from treatment with phenethyl isothiocyanate.

### Example 3

An aqueous solution of 3-aminobutanoic acid was applied to flowering Arabidopsis thaliana plants to enhance the frequency of meiotic recombination (crossovers). Silwet, a spraying adjuvant, was added to the 3-aminobutanoic acid solution at a volume/volume concentration of 0.01%. The solution was applied to the plants by foliar spraying until the plants were drenched. Several concentrations of 3-aminobutanoic acid were used including 0.1, 1, 7.5, 10, 12.5 and 15 mM. As a control, other plants (i.e., control plants) were treated with an aqueous 0.01% solution of Silwet by foliar spraying the solution on the plants until the plants were drenched.

Crossover frequencies were measured in pollen from treated and control plants using a visual fluorescent pollen transgene assay as described in Francis et al., Proc Natl Acad Sci USA. 2007; 104 (10): 3913-3918. In this assay pollen tetrads are scored as Parental Ditype (PD), Nonparental Ditype (NPD) or Tetratype (TT) and the frequency of those tetrad classes are used to calculate a genetic map distances in centiMorgans (cM) between reporter genes expressing fluorescent proteins. The genetic map distances between a pair of markers on chromosome 5 were compared between control plants and treated plants. The genetic interval between the two markers was designated as I5d. All concentrations of 3-aminobutanoic acid were found to increase meiotic recombination compared in treated plants compared to controls as shown Table 2. In Table 2, each experimental treatment is paired with data from control plants that were treated and scored together and designated with an experiment number in the first column.

TABLE 2

| Exp. | Treatment | | PD | NPD | TT | cM | p-value |
|---|---|---|---|---|---|---|---|
| (1) | Water | | 928 | 0 | 124 | 5.89 | |
| (2) | Water | | 964 | 0 | 116 | 5.37 | |
| (3) | Water | | 955 | 0 | 118 | 5.50 | |
| (3) | 15 | mM | 849 | 2 | 160 | 8.51 | 0.000 |
| (3) | 12.5 | mM | 908 | 4 | 166 | 8.81 | 0.000 |
| (1) | 10 | mM | 1092 | 0 | 221 | 8.42 | 0.000 |
| (2) | 1 | mM | 983 | 0 | 169 | 7.34 | 0.003 |
| (1) | 0.1 | mM | 926 | 0 | 178 | 8.06 | 0.002 |

These results were validated by scoring a second pair of markers on chromosome 2 designated as I2b, using 3 concentrations of 3-aminobutanoic acid. All three concentrations increased meiotic recombination in treated plants compared to control plants as shown in Table 3.

TABLE 3

| Exp. | Treatment | | PD | NPD | TT | cM | p-value |
|---|---|---|---|---|---|---|---|
| (1) | Water | | 861 | 0 | 98 | 5.11 | |
| (1) | 12.5 | mM | 865 | 0 | 155 | 7.60 | 0.000 |
| (1) | 10 | mM | 866 | 1 | 140 | 7.25 | 0.003 |
| (1) | 7.5 | mM | 867 | 3 | 128 | 7.31 | 0.006 |

### Example 4

An aqueous solution phenethyl isothiocyanate (IUPAC name: 2-isothiocyanatoethylbenzene) was applied to flowering Arabidopsis thaliana plants to enhance the frequency of meiotic recombination (crossovers). Phenethyl isothiocyanate in an aqueous solution containing 10% DMSO and 0.01% Silwet was applied by foliar spray until the plants were drenched. The phenethyl isothiocyanate was first dissolved in 100% DMSO and then diluted to its final concentration with 0.0.1% Silwet in water. Several concentrations of phenethyl isothiocyanate were used including 7.5, 10, and 12.5 mM. Control plants were treated with an aqueous solution of 10% DMSO and 0.01% Silwet by foliar spraying the solution on the control plants until the control plants were drenched.

Crossover frequencies were measured in pollen from treated and control plants using a visual fluorescent pollen transgene assay as described in Francis et al., Proc Natl Acad Sci USA. 2007; 104 (10): 3913-3918. In this assay pollen tetrads are scored as Parental Ditype (PD), Nonparental Ditype (NPD) or Tetratype (TT) and the frequency of those tetrad classes are used to calculate a genetic map distances in centiMorgans (cM) between reporter genes expressing fluorescent proteins. The genetic map distances between a pair of markers on chromosome 5 were compared between control plants and treated plants. The genetic interval between the two markers was designated as I5d. All concentrations of phenethyl isothiocyanate were found to increase meiotic recombination compared in treated plants compared to controls as shown in Table 4. In Table 4, each experimental treatment is paired with data from control plants that were treated and scored together and designated with an experiment number in the first column.

TABLE 4

| Exp. | Treatment | | PD | NPD | TT | cM | p-value |
|---|---|---|---|---|---|---|---|
| (1) | DMSO | | 889 | 0 | 116 | 5.77 | |
| (2) | DMSO | | 942 | 0 | 126 | 5.90 | |
| (3) | DMSO | | 842 | 1 | 114 | 6.27 | |
| (2) | 12.5 | mM | 868 | 0 | 200 | 9.36 | 0.000 |
| (1) | 10 | mM | 1840 | 0 | 370 | 8.37 | 0.000 |
| (2) | 7.5 | mM | 978 | 2 | 211 | 9.36 | 0.000 |

These results were validated by scoring a second pair of markers on chromosome 2 designated as I2a, using 3 concentrations of 3-aminobutanoic acid: 2.5, 7.5 and 10 mM. Two of the three concentrations, 2.5 and 7.5 mM, increased meiotic recombination in treated plants compared to control plants as shown in Table 5.

TABLE 5

| | | | | | | |
|---|---|---|---|---|---|---|
| Exp. | Treatment | PD | NPD | TT | cM | p-value |

| | | | | | |
|---|---|---|---|---|---|
| (1) | DMSO | 944 | 0 | 46 | 2.32 |

| | | | | | | | |
|---|---|---|---|---|---|---|---|
| (1) | 10 | mM | 941 | 0 | 60 | 3.00 | 0.090 |
| (1) | 7.5 | mM | 914 | 0 | 66 | 3.37 | 0.023 |
| (1) | 2.5 | mM | 924 | 0 | 67 | 3.38 | 0.021 |

### Example 5

An aqueous solution BIX-01294 (IUPAC name: N-(1-benzylpiperidin-4-yl)-6,7-dimethoxy-2-(4-methyl-1,4-diazepan-1-yl) quinazolin-4-amine) was applied to flowering Arabidopsis thaliana plants to enhance the frequency of meiotic recombination (crossovers). BIX-01294 in an aqueous solution containing 10% DMSO and 0.01% Silwet was applied by foliar spray until the plants were drenched. The BIX-01294 was first dissolved in 100% DMSO and then diluted to its final concentration with 0.0.1% Silwet in water. Several concentrations of phenethyl isothiocyanate were used including 0.5, 1, 2.5 and 5 mM. Control plants were treated with an aqueous solution of 10% DMSO and 0.01% Silwet by foliar spraying the solution on the control plants until the control plants were drenched.

Crossover frequencies were measured in pollen from treated and control plants using a visual fluorescent pollen transgene assay as described in Francis et al., Proc Natl Acad Sci USA. 2007; 104 (10): 3913-3918. In this assay pollen tetrads are scored as Parental Ditype (PD), Nonparental Ditype (NPD) or Tetratype (TT) and the frequency of those tetrad classes are used to calculate a genetic map distances in centiMorgans (cM) between reporter genes expressing fluorescent proteins. The genetic map distances between a pair of markers on chromosome 5 were compared between control plants and treated plants. The genetic interval between the two markers was designated as I5d. Three of the four concentrations, 0.5, 2.5 and 5 mM, of BIX-01294 were found to increase meiotic recombination compared in treated plants compared to controls as shown in Table 6. In Table 6, each experimental treatment is paired with data from control plants that were treated and scored together and designated with an experiment number in the first column.

TABLE 6

| Exp. | Treatment | | PD | NPD | TT | cM | p-value |
|---|---|---|---|---|---|---|---|
| (1) | DMSO | | 917 | 0 | 126 | 6.04 | |
| (2) | DMSO | | 797 | 0 | 124 | 6.73 | |
| (3) | DMSO | | 893 | 0 | 106 | 5.31 | |
| (3) | 5 | mM | 938 | 4 | 156 | 8.20 | 0.001 |
| (3) | 2.5 | mM | 888 | 5 | 169 | 9.37 | 0.000 |
| (2) | 1 | mM | 829 | 0 | 131 | 6.82 | 0.454 |
| (1) | 0.5 | mM | 936 | 1 | 185 | 8.51 | 0.001 |

These results were validated by scoring a second pair of markers on chromosome 2 designated as I2a, using five concentrations of BIX-01294:0.1, 0.5, 1, 2.5 and 5 mM. Three of the five concentrations, 0.1, 0.5, and 1 mM, increased meiotic recombination in treated plants compared to control plants as shown in Table 7.

TABLE 7

| | | | | | | |
|---|---|---|---|---|---|---|
| Exp. | Treatment | PD | NPD | TT | cM | p-value |

| | | | | | |
|---|---|---|---|---|---|
| (1) | DMSO | 915 | 0 | 42 | 2.19 |
| (2) | DMSO | 944 | 0 | 55 | 2.75 |

| | | | | | | | |
|---|---|---|---|---|---|---|---|
| (2) | 5 | mM | 836 | 0 | 43 | 2.45 | 0.725 |
| (2) | 2.5 | mM | 777 | 0 | 55 | 3.31 | 0.163 |
| (1) | 1 | mM | 988 | 0 | 99 | 4.55 | 0.000 |
| (1) | 0.5 | mM | 922 | 0 | 84 | 4.17 | 0.000 |
| (1) | 0.1 | mM | 908 | 0 | 59 | 3.05 | 0.046 |

The foregoing is illustrative of the present invention, and is not to be construed as limiting thereof. The invention is defined by the following claims, with equivalents of the claims to be included therein.

1. A method of modifying meiotic recombination in a plant, the method comprising:
applying a compound that modulates meiotic recombination to the plant, thereby modifying meiotic recombination in the plant.

2. The method of claim 1, wherein the method increases the frequency of meiotic recombination in the plant, optionally wherein the frequency of meiotic recombination in the plant is compared to the frequency of meiotic recombination in a control plant and/or a parent plant and/or wherein the frequency of meiotic recombination is measured using a pollen tetrad-based visual assay, by scoring the segregation of loci whose expression results in observable phenotypes, and/or by genotyping (e.g., genotyping the gametes and/or pollen of the plant); or
wherein the method decreases the frequency of meiotic recombination in the plant, optionally wherein the frequency of meiotic recombination in the plant is compared to the frequency of meiotic recombination in a control plant and/or a parent plant and/or wherein the frequency of meiotic recombination is measured using a pollen tetrad-based visual assay, by scoring the segregation of loci whose expression results in observable phenotypes, by nucleic acid sequencing, and/or by genotyping (e.g., genotyping the gametes and/or pollen of the plant).

3. (canceled)

4. The method of claim 1, wherein the method is devoid of genetic engineering to modify meiotic recombination in the plant.

5. The method of claim 1, wherein the method increases expression and/or activity of a nucleic acid and/or protein in the plant; or
wherein the method decreases expression and/or activity of a nucleic acid and/or protein in the plant.

6. (canceled)

7. The method of claim 1, wherein applying the compound that modulates meiotic recombination to the plant comprises exogenously contacting the compound that modulates meiotic recombination to at least a portion of the plant; optionally spraying the compound that modulates meiotic recombination onto at least a portion of the plant, soaking at least a portion of the plant in a composition comprising the compound that modulates meiotic recombination, and/or contacting soil adjacent to the plant with the compound that modulates meiotic recombination, optionally wherein contacting soil adjacent to the plant with the compound that modulates meiotic recombination comprises soil drenching with the compound that modulates meiotic recombination.

8.-10. (canceled)

11. The method of claim 1, wherein the compound that modulates meiotic recombination is a compound that mimics temperature shock in the plant, a compound that mimics pathogen stress in the plant, an allelopathic compound, a compound that inhibits non-homologous end joining (NHEJ) in the plant, a compound that affects helicase activity and/or formation in the plant, a compound that affects epigenetic marks and/or epigenetic modifiers, and/or a compound that affects a component used in a meiotic recombination pathway in the plant.

12. The method of claim 1, wherein the compound that modulates meiotic recombination is selected from the group consisting of: ethyl 4-[(1-hydroxy-2-phenylindol-3-yl)-pyridin-2-ylmethyl]piperazine-1-carboxylate, N-(5-tert-butyl-1H-pyrazol-3-yl)-2-[(3R)-3-propan-2-ylpiperazin-1-yl]-7H-pyrrolo[2,3-d]pyrimidin-4-amine, N-(6-aminohexyl)-5-chloro-1-naphthalenesulfonamide, 2-chloro-10-(3-dimethylaminopropyl) phenothiazine hydrochloride, Z-5-(4-hydroxybenzylidene)-2-imino-1,3-thiazolidin-4-one, 2-[(2R)-2-methylpyrrolidin-2-yl]-1H-benzimidazole-4-carboxamide, 5-[(E)-2-(4-hydroxyphenyl) ethenyl]benzene-1,3-diol, 3-(benzylsulfamoyl)-4-bromo-N-(4-bromophenyl)benzamide, 2-(4-Morpholinyl)-6-(1-thianthrenyl)-4H-Pyran-4-one, an azane, a dichloroplatinum (2+) compound, cisplatin, 2-(pyridin-3-ylmethylidene) indene-1,3-dione, [(1S,2S,4R)-4-[4-[(1S)-2,3-dihydro-1H-inden-1-yl]amino]pyrrolo[2,3-d]pyrimidin-7-yl]-2-hydroxycyclopentyl]methyl sulfamate, 2-[2-(3,4-dimethoxyphenyl)ethylamino]-6-(3-fluorophenyl)-8H-pyrimido[4,5-d]pyrimidine-5,7-dione, 1-(4-((dimethylamino)methyl)phenyl)-8,9-dihydro-2,7,9a-triazabenzo[cd]azulen-6 (7H)-one, (2E,4E,6R)-7-[4-(dimethylamino)phenyl]-N-hydroxy-4,6-dimethyl-7-oxohepta-2,4-dienamide, 4-[(E)-2-(3,5-dimethoxyphenyl) ethenyl]phenol, 4-amino-N-(4,6-dimethylpyrimidin-2-yl)benzenesulfonamide, (2S)-2-amino-4-ethylsulfanylbutanoic acid, 4-amino-1-[(2R,3R,4S,5R)-3,4-dihydroxy-5-(hydroxymethyl)oxolan-2-yl]-1,3,5-triazin-2-one, sodium butanoate, 5,7-dihydroxy-3-(4-hydroxyphenyl) chromen-4-one, pyridine-2,4-dicarboxylic acid, (1R,2S)-2-phenylcyclopropan-1-amine, 4-amino-1-[(2R,4S,5R)-4-hydroxy-5-(hydroxymethyl)oxolan-2-yl]-1,3,5-triazin-2-one, 1-[(2R,3R,4S,5R)-3,4-dihydroxy-5-(hydroxymethyl)oxolan-2-yl]pyrimidin-2-one, 3-[4-[(1R,2S)-2-aminocyclopropyl]phenyl]phenol, methyl 3-[4-(4-carbamimidoylbenzoyl) piperazine-1-carbonyl]-5-[(4-carbamimidoylpiperazin-1-yl)methyl]benzoate, 2-[[2-[2-(dimethylamino)ethyl-ethylamino]-2-oxoethyl]amino]methyl]pyridine-4-carboxamide, 2-(4-methylphenyl)-1,2-benzothiazol-3-one, 5-chloro-N-[(E)-[phenyl(pyridin-2-yl)methylidene]amino]pyridin-2-amine, methyl 2-benzamido-1-(3-phenylpropyl)benzimidazole-5-carboxylate, 2-[(2-hydroxynaphthalen-1-yl)methylideneamino]-N-(1-phenylethyl)benzamide, 3-(prop-2-enyldisulfanyl) prop-1-ene, N-(1-benzylpiperidin-4-yl)-6,7-dimethoxy-2-(4-methyl-1,4-diazepan-1-yl) quinazolin-4-amine, 5′-methoxy-6′-(3-pyrrolidin-1-ylpropoxy)spiro[cyclobutane-1,3′-indole]-2′-amine, 2-cyclohexyl-6-methoxy-N-(1-propan-2-ylpiperidin-4-yl)-7-(3-pyrrolidin-1-ylpropoxy) quinazolin-4-amine, 7-[3-(dimethylamino) propoxy]-6-methoxy-2-(4-methyl-1,4-14-(hydroxymethyl)-3-[14-diazepan-1-yl)-N-(1-methylpiperidin-4-yl) quinazolin-4-amine, (hydroxymethyl)-18-methyl-13,17-dioxo-15,16-dithia-10,12,18-triazapentacyclo[12.2.2.01,12.03,11.04,9]octadeca-4,6,8-trien-3-yl]-18-methyl-15,16-dithia-10,12,18-triazapentacyclo[12.2.2.01, 12.03,11.04,9]octadeca-4,6,8-triene-13,17-dione, 2,3-dichloro-1,4-naphthoquinone, 2,3-dichloro-5,8-dihydroxy-1,4-naphthoquinone, (1S,2R,4S)-1,7,7-trimethylbicyclo[2.2.1]heptan-2-ol, 2-isothiocyanatoethylbenzene, 4-isothiocyanatobutylbenzene, 3-isothiocyanatoprop-1-ene, isothiocyanatoethane, 1-isothiocyanatobutane, 3-isothiocyanato-2-methylprop-1-ene, [(4E,6Z,8S,9S,10E,12S, 13R, 14S, 16R)-13-hydroxy-8, 14, 19-trimethoxy-4, 10,12, 16-tetramethyl-3,20,22-trioxo-2-azabicyclo[16.3.1]docosa-1 (21),4,6,10,18-pentaen-9-yl]carbamate, 4-[[(2R,3S,4R,5R)-5-[6-amino-8-[(3,4-dichlorophenyl)methylamino]purin-9-yl]-3,4-dihydroxyoxolan-2-yl]methoxymethyl]benzonitrile, 24-methyl-5,7, 18,20-tetraoxa-24-azoniahexacyclo[11.11.0.02,10.04,8.014,22.017,21]tetracosa-1 (24),2,4 (8),9,11,13,15,17 (21),22-nonaene, (4S)-4-prop-1-en-2-ylcyclohexene-1-(1R,4R)-1,7,7-carbaldehyde, (4R)-1-methyl-4-prop-1-en-2-ylcyclohexene, trimethylbicyclo[2.2.1]heptan-2-one, 1,3,3-trimethyl-2-oxabicyclo[2.2.2]octane, 2,6,6-trimethylbicyclo[3.1.1]hept-2-ene, isothiocyanatobenzene, 2-isothiocyanatopropane, 1-isothiocyanato-2-methylpropane, 1-isothiocyanatohexane, 1-isothiocyanato-4-methylsulfinylbutane, 1-isothiocyanato-5-methylsulfinylpentane, 8-[(6-iodo-1,3-benzodioxol-5-yl)sulfanyl]-9-[3-(propan-2-ylamino) propyl]purin-6-amine, [4-[2-carbamoyl-5-[6,6-dimethyl-4-oxo-3-(trifluoromethyl)-5,7-dihydroindazol-1-yl]anilino]cyclohexyl]2-aminoacetate, 3-(2,4-dihydroxy-5-propan-2-ylphenyl)-4-(1-methylindol-5-yl)-1H-1,2,4-triazol-5-one, N-acetyl-5-methoxytryptamine, S-methyl 1,2,3-benzothiadiazole-7-carbothioate, 5-chloro-2-methylpyrazole-3-carboxylic acid, 3-prop-2-enoxy-1,2-benzothiazole 1,1-dioxide, 3-aminobutanoic acid, 1,1-dioxo-1,2-benzothiazol-3-one, 4-hydroxybenzohydrazide, 3-hydroxy-3-(2-oxopropyl)-1H-indol-2-one, 2-[3-[(4-amino-2-methylpyrimidin-5-yl)methyl]-4-methyl-1,3-thiazol-3-ium-5-yl]ethanol, 2,6-dichloropyridine-4-carboxylic acid, 3-methoxybenzo[d]isothiazole 1,1-dioxide, N-(3-chloro-4-methylphenyl)-4-methylthiadiazole-5-carboxamide, 3,4-dichloro-N-(2-cyanophenyl)-1,2-thiazole-5-carboxamide, 2-amino-3,5-dichlorobenzoic acid, 3-(furan-2-yl)-3-phenylpropan-1-amine, 4-amino-N-(5-methoxypyrimidin-2-yl)benzenesulfonamide, 4-amino-N-(6-methoxypyridazin-3-yl)benzenesulfonamide, N-(4-aminophenyl)sulfonylbenzamide, 4-amino-N-(6-chloropyridazin-3-yl)benzenesulfonamide, 4-amino-N-(5-methyl-1,2-oxazol-3-yl)benzenesulfonamide, 3-(butylamino)-4-phenoxy-5-sulfamoylbenzoic acid, 3-benzyl-1,1-dioxo-6-(trifluoromethyl)-3,4-dihydro-2H-126,2,4-benzothiadiazine-7-sulfonamide, 4-chloro-N-[(2R,6S)-2,6-dimethylpiperidin-1-yl]-3-sulfamoylbenzamide, (3R,4S,5R,6R)-4-[(3R,4S,5R,6R)-3,5-dihydroxy-6-(hydroxymethyl)-4-[(3R,4S,5S,6R)-3,4,5-trihydroxy-6-(hydroxymethyl) oxan-2-yl]oxyoxan-2-yl]oxy-6-(hydroxymethyl) oxane-2,3,5-triol, (3R,6S)-3-hydroxy-3-[(3-hydroxyphenyl)methyl]-1,4-dimethyl-6-[(4-nitro-1H-indol-3-yl)methyl]piperazine-2,5-dione, 2,4-dichloro-6-[(3-methoxyphenyl)iminomethyl]phenol, 1,2-benzothiazole 1,1-dioxide, 5-[(3-carboxy-4-hydroxyphenyl)-(3-carboxy-4-oxocyclohexa-2,5-dien-1-ylidene)methyl]-2-hydroxybenzoic acid, disodium 6-methyl-2-[4-[2-[4-(6-methyl-7-sulfonato-1,3-benzothiazol-2-yl)phenyl]iminohydrazinyl]phenyl]-1,3-benzothiazole-7-sulfonate, 8-[[4-methyl-3-[[3-[[3-[[2-methyl-5-[(4,6,8-trisulfonaphthalen-1-yl)carbamoyl]phenyl]carbamoyl]phenyl]carbamoylamino]benzoyl]amino]benzoyl]amino]nap hthalene-1,3,5-trisulfonic acid, 1-[4-Fluoro-3-(trifluoromethyl)phenyl]-3-(5-pyridin-4-yl-1,3,4-thiadiazol-2-yl) urea, 3-[18-(2-carboxyethyl)-7,12-diethyl-3,8,13,17,22-pentamethyl-23H-porphyrin-2-yl]propanoic acid, 1h-pyrrole-2,5-dione, 1-[(1-oxopropoxy)methyl]-N-propionyloxymethyl-maleimide, 3,4-dichloro-1-[3-(3,4-dichloro-2,5-dioxopyrrol-1-yl)-2,2-dimethylpropyl]pyrrole-2,5-dione, 5,7-dioxa-12-azoniapentacyclo[10.6.1.02,10.04,8.015,19]nonadeca-1 (18),2,4 (8),9,11,15 (19), 16-heptaen-17-ol, 5,6-bis(((E)-benzylidene)amino)-2-thioxo-2,3-dihydropyrimidin-4 (1H)-one, (6Z)-3,7,11-trimethyldodeca-1,6,10-trien-3-ol, (2S,5R)-5-methyl-2-propan-2-ylcyclohexan-1-one, (1S,2S,5S,8R,9S,10S,11R, 15S,18R)-9,10, 15,18-tetrahydroxy-12,12-dimethyl-6-methylidene-17-oxapentacyclo[7.6.2.15,8.01,11.02,8]octadecan-7-one, (1R,2R,5R,9R,12R,16R)-5-ethenyl-1,5,12-trimethyl-10-oxatetracyclo[7.6.1.02,7.012, 16]hexadec-7-ene-11,13-dione, (1S,2R,5R, 12R, 18R)-5-ethenyl-13-hydroxy-5,12-dimethyl-10,14-dioxapentacyclo[11.2.2.11,9.02,7.012,18]octadec-7-en-11-one, [(1R,2R,4S,6S,9R, 10S, 13S, 16R)-2, 16-dihydroxy-5,5,9-trimethyl-14-methylidene-15-oxo-6-tetracyclo[11.2.1.01,10.04,9]hexadecanyl]acetate, (1S,4S,8R,9R,12S, 13S, 14S, 16S)-9,14-dihydroxy-7,7-dimethyl-17-methylidene-3,10-dioxapentacyclo[14.2.1.01,13.04,12.08,12]nonadecane-2,18-dione, [(1S,1′R,3'S,5R,6S, 7S,9S)-3′-acetyloxy-7-hydroxy-6′,6′-dimethyl-10-methylidene-2,11-dioxospiro[3-oxatricyclo[7.2.1.01,6]dodecane-5,2′-cyclohexane]-1′-yl]methyl acetate, (3E,6E)-3,7,11-trimethyldodeca-1,3,6,10-tetraene, 6,6-dimethyl-2-methylidenebicyclo[3.1.1]heptane, 2,2-dimethyl-3-methylidenebicyclo[2.2.1]heptane, 5-methyl-2-propan-2-ylphenol, (2E)-3,7-dimethylocta-2,6-dien-1-ol, (2E)-3,7-dimethylocta-2,6-dienal, 5-methyl-2-propan-2-ylcyclohexan-1-ol, benzoic acid, 2-aminophenoxazin-3-one, 2-amino-7-methoxyphenoxazin-3-one, (2S,3R)-2-(3,4-dihydroxyphenyl)-3,4-dihydro-2H-chromene-3,5,7-triol, and any combination thereof.

13.-21. (canceled)

22. The method of claim 1, wherein the compound that modulates meiotic recombination is present in a composition and applying the compound that modulates meiotic recombination to the plant comprises applying the composition to the plant; optionally wherein the composition is a liquid, optionally wherein the liquid comprises water, dimethylsulfoxide, and/or a surfactant; optionally wherein the compound that modulates meiotic recombination is present in the composition in an amount of about 0.001 or 0.01 mM to about 20 mM.

23.-24. (canceled)

25. The method of claim 1, wherein applying the compound that modulates meiotic recombination to the plant comprises:
applying the compound that modulates meiotic recombination to the plant at a time prior to, during, and/or after the transition from the vegetative phase to the reproductive phase of the plant, optionally wherein the compound is applied to the plant during the plant's reproductive phase;
applying the compound that modulates meiotic recombination to a cell of the plant, wherein the cell is a meiocyte and/or is undergoing meiosis, optionally wherein the cell is a vegetative cell;
applying the compound that modulates meiotic recombination to the sporophyte generation of the plant to modulate meiotic recombination in the gametophyte generation, optionally wherein the gametophyte generation has an increased number of crossovers compared to the gametophyte generation generated in the absence of a method of the present invention and/or to the parent generation generated in the absence of a method of the present invention;
applying the compound that modulates meiotic recombination to a reproductive part of the plant (e.g., flower bud, inflorescence, flower, stamen, pistil, etc.); and/or
applying the compound that modulates meiotic recombination to the aerial parts of the plant.

26.-29. (canceled)

30. The method of claim 1, wherein the method improves (e.g., reduces) linkage drag in the plant and/or its progeny.

31. The method of claim 1, wherein the method eliminates the need for back crossing or decreases the amount of time for back crossing for the plant and/or its progeny, optionally wherein the method of devoid of a back crossing step.

32. The method of claim 1, wherein the plant is a crop plant, a monocot, or a dicot, optionally a eudicot.

33.-34. (canceled)

35. A method of reducing linkage drag in a plant, increasing recombination in a cold region of a plant genome, and/or reducing the number of backcross generations in a plant breeding method, the method comprising:
applying a compound that modulates meiotic recombination to a plant, thereby reducing linkage drag in the plant, increasing recombination in a cold region of the genome of the plant, and/or reducing the number of backcross generations in a plant breeding method including the plant.

36. The method of claim 35, wherein the method increases the frequency of meiotic recombination in the plant, optionally wherein the frequency of meiotic recombination in the plant is compared to the frequency of meiotic recombination in a control plant and/or a parent plant and/or wherein the frequency of meiotic recombination is measured using a pollen tetrad-based visual assay, by scoring the segregation of loci whose expression results in observable phenotypes, by nucleic acid sequencing, and/or by genotyping (e.g., genotyping the gametes and/or pollen of the plant); or
wherein the method decreases the frequency of meiotic recombination in the plant, optionally wherein the frequency of meiotic recombination in the plant is compared to the frequency of meiotic recombination in a control plant and/or a parent plant and/or wherein the frequency of meiotic recombination is measured using a pollen tetrad-based visual assay, by scoring the segregation of loci whose expression results in observable phenotypes, by nucleic acid sequencing, and/or by genotyping (e.g., genotyping the gametes and/or pollen of the plant).

37. (canceled)

38. The method of claim 35, wherein the method is devoid of genetic engineering to modify meiotic recombination in the plant.

39. The method of claim 35, wherein the method increases expression and/or activity of a nucleic acid and/or protein in the plant; or decreases expression and/or activity of a nucleic acid and/or protein in the plant.

40. (canceled)

41. The method of claim 35, wherein applying the compound that modulates meiotic recombination to the plant comprises exogenously contacting the compound that modulates meiotic recombination to at least a portion of the plant; optionally spraying the compound that modulates meiotic recombination onto at least a portion of the plant, soaking at least a portion of the plant in a composition comprising the compound that modulates meiotic recombination, and/or contacting soil adjacent to the plant with the compound that modulates meiotic recombination, optionally wherein contacting soil adjacent to the plant with the compound that modulates meiotic recombination comprises soil drenching with the compound that modulates meiotic recombination.

42-44. (canceled)

45. The method of claim 35, wherein the compound that modulates meiotic recombination is a compound that mimics temperature shock in the plant, a compound that mimics pathogen stress in the plant, an allelopathic compound, a compound that inhibits non-homologous end joining (NHEJ) in the plant, a compound that affects helicase activity and/or formation in the plant, a compound that affects epigenetic marks and/or epigenetic modifiers, and/or a compound that affects a component used in a meiotic recombination pathway in the plant.

46. The method of claim 35, wherein the compound that modulates meiotic recombination is selected from the group consisting of: ethyl 4-[(1-hydroxy-2-phenylindol-3-yl)-pyridin-2-ylmethyl]piperazine-1-carboxylate, N-(5-tert-butyl-1H-pyrazol-3-yl)-2-[(3R)-3-propan-2-ylpiperazin-1-yl]-7H-pyrrolo[2,3-d]pyrimidin-4-amine, N-(6-aminohexyl)-5-chloro-1-naphthalenesulfonamide, 2-chloro-10-(3-dimethylaminopropyl) phenothiazine hydrochloride, Z-5-(4-hydroxybenzylidene)-2-imino-1,3-thiazolidin-4-one, 2-[(2R)-2-methylpyrrolidin-2-yl]-1H-benzimidazole-4-carboxamide, 5-[(E)-2-(4-hydroxyphenyl) ethenyl]benzene-1,3-diol, 3-(benzylsulfamoyl)-4-bromo-N-(4-bromophenyl)benzamide, 2-(4-Morpholinyl)-6-(1-thianthrenyl)-4H-Pyran-4-one, an azane, a dichloroplatinum (2+) compound, cisplatin, 2-(pyridin-3-ylmethylidene) indene-1,3-dione, [(1S,2S,4R)-4-[4-[[(1S)-2,3-dihydro-1H-inden-1-yl]amino]pyrrolo[2,3-d]pyrimidin-7-yl]-2-hydroxycyclopentyl]methyl sulfamate, 2-[2-(3,4-dimethoxyphenyl)ethylamino]-6-(3-fluorophenyl)-8H-pyrimido[4,5-d]pyrimidine-5,7-dione, 1-(4-((dimethylamino)methyl)phenyl)-8,9-dihydro-2,7,9a-triazabenzo[cd]azulen-6 (7H)-one, (2E,4E,6R)-7-[4-(dimethylamino)phenyl]-N-hydroxy-4,6-dimethyl-7-oxohepta-2,4-dienamide, 4-[(E)-2-(3,5-dimethoxyphenyl) ethenyl]phenol, 4-amino-N-(4,6-dimethylpyrimidin-2-yl)benzenesulfonamide, (2S)-2-amino-4-ethylsulfanylbutanoic acid, 4-amino-1-[(2R,3R,4S,5R)-3,4-dihydroxy-5-(hydroxymethyl)oxolan-2-yl]-1,3,5-triazin-2-one, sodium butanoate, 5,7-dihydroxy-3-(4-hydroxyphenyl) chromen-4-one, pyridine-2,4-dicarboxylic acid, (1R,2S)-2-phenylcyclopropan-1-amine, 4-amino-1-[(2R,4S,5R)-4-hydroxy-5-(hydroxymethyl)oxolan-2-yl]-1,3,5-triazin-2-one, 1-[(2R,3R,4S,5R)-3,4-dihydroxy-5-(hydroxymethyl)oxolan-2-yl]pyrimidin-2-one, 3-[4-[(1R,2S)-2-aminocyclopropyl]phenyl]phenol, methyl 3-[4-(4-carbamimidoylbenzoyl) piperazine-1-carbonyl]-5-[(4-carbamimidoylpiperazin-1-yl)methyl]benzoate, 2-[[[2-[2-(dimethylamino)ethyl-ethylamino]-2-oxoethyl]amino]methyl]pyridine-4-carboxamide, 2-(4-methylphenyl)-1,2-benzothiazol-3-one, 5-chloro-N-[(E)-[phenyl(pyridin-2-yl)methylidene]amino]pyridin-2-amine, methyl 2-benzamido-1-(3-phenylpropyl)benzimidazole-5-carboxylate, 2-[(2-hydroxynaphthalen-1-yl)methylideneamino]-N-(1-phenylethyl)benzamide, 3-(prop-2-enyldisulfanyl) prop-1-ene, N-(1-benzylpiperidin-4-yl)-6,7-dimethoxy-2-(4-methyl-1,4-diazepan-1-yl) quinazolin-4-amine, 5′-methoxy-6′-(3-pyrrolidin-1-ylpropoxy)spiro[cyclobutane-1,3′-indole]-2′-amine, 2-cyclohexyl-6-methoxy-N-(1-propan-2-ylpiperidin-4-yl)-7-(3-pyrrolidin-1-ylpropoxy) quinazolin-4-amine, 7-[3-(dimethylamino) propoxy]-6-methoxy-2-(4-methyl-1,4-diazepan-1-yl)-N-(1-methylpiperidin-4-yl) quinazolin-4-amine, 14-(hydroxymethyl)-3-[14-(hydroxymethyl)-18-methyl-13,17-dioxo-15,16-dithia-10,12,18-triazapentacyclo[12.2.2.01,12.03,11.04,9]octadeca-4,6,8-trien-3-yl]-18-methyl-15,16-dithia-10,12,18-triazapentacyclo[12.2.2.01, 12.03, 11.04,9]octadeca-4,6,8-triene-13,17-dione, 2,3-dichloro-1,4-naphthoquinone, 2,3-dichloro-5,8-dihydroxy-1,4-naphthoquinone, (1S,2R,4S)-1,7,7-trimethylbicyclo[2.2.1]heptan-2-ol, 2-isothiocyanatoethylbenzene, 4-isothiocyanatobutylbenzene, 3-isothiocyanatoprop-1-ene, isothiocyanatoethane, 1-isothiocyanatobutane, 3-isothiocyanato-2-methylprop-1-ene, [(4E,6Z,8S,9S, 10E, 12S, 13R, 14S, 16R)-13-hydroxy-8, 14, 19-trimethoxy-4, 10, 12, 16-tetramethyl-3,20,22-trioxo-2-azabicyclo[16.3.1]docosa-1 (21),4,6, 10,18-pentaen-9-yl]carbamate, 4-[[(2R,3S,4R,5R)-5-[6-amino-8-[(3,4-dichlorophenyl)methylamino]purin-9-yl]-3,4-dihydroxyoxolan-2-yl]methoxymethyl]benzonitrile, 24-methyl-5,7, 18,20-tetraoxa-24-azoniahexacyclo[11.11.0.02,10.04,8.014,22.017,21]tetracosa-1 (24),2,4 (8),9,11,13,15,17 (21),22-nonaene, (4S)-4-prop-1-en-2-ylcyclohexene-1-carbaldehyde, (4R)-1-methyl-4-prop-1-en-2-ylcyclohexene, (1R,4R)-1,7,7-trimethylbicyclo[2.2.1]heptan-2-one, 1,3,3-trimethyl-2-oxabicyclo[2.2.2]octane, 2,6,6-trimethylbicyclo[3.1.1]hept-2-ene, isothiocyanatobenzene, 2-isothiocyanatopropane, 1-isothiocyanato-2-methylpropane, 1-isothiocyanatohexane, 1-isothiocyanato-4-methylsulfinylbutane, 1-isothiocyanato-5-methylsulfinylpentane, 8-[(6-iodo-1,3-benzodioxol-5-yl)sulfanyl]-9-[3-(propan-2-ylamino) propyl]purin-6-amine, [4-[2-carbamoyl-5-[6,6-dimethyl-4-oxo-3-(trifluoromethyl)-5,7-dihydroindazol-1-yl]anilino]cyclohexyl]2-aminoacetate, 3-(2,4-dihydroxy-5-propan-2-ylphenyl)-4-(1-methylindol-5-yl)-1H-1,2,4-triazol-5-one, N-acetyl-5-methoxytryptamine, S-methyl 1,2,3-benzothiadiazole-7-carbothioate, 5-chloro-2-methylpyrazole-3-carboxylic acid, 3-prop-2-enoxy-1,2-benzothiazole 1,1-dioxide, 3-aminobutanoic acid, 1,1-dioxo-1,2-benzothiazol-3-one, 4-hydroxybenzohydrazide, 3-hydroxy-3-(2-oxopropyl)-1H-indol-2-one, 2-[3-[(4-amino-2-methylpyrimidin-5-yl)methyl]-4-methyl-1,3-thiazol-3-ium-5-yl]ethanol, 2,6 dichloropyridine-4-carboxylic acid, 3-methoxybenzo[d]isothiazole 1,1-dioxide, N-(3-chloro-4-methylphenyl)-4-methylthiadiazole-5-carboxamide, 3,4-dichloro-N-(2-cyanophenyl)-1,2-thiazole-5-carboxamide, 2-amino-3,5-dichlorobenzoic acid, 3-(furan-2-yl)-3-phenylpropan-1-amine, 4-amino-N-(5-methoxypyrimidin-2-yl)benzenesulfonamide, 4-amino-N-(6-methoxypyridazin-3-yl)benzenesulfonamide, N-(4-aminophenyl)sulfonylbenzamide, 4-amino-N-(6-chloropyridazin-3-yl)benzenesulfonamide, 4-amino-N-(5-methyl-1,2-oxazol-3-yl)benzenesulfonamide, 3-(butylamino)-4-phenoxy-5-sulfamoylbenzoic acid, 3-benzyl-1,1-dioxo-6-(trifluoromethyl)-3,4-dihydro-2H-126,2,4-benzothiadiazine-7-sulfonamide, 4-chloro-N-[(2R,6S)-2,6-dimethylpiperidin-1-yl]-3-sulfamoylbenzamide, (3R,4S,5R,6R)-4-[(3R,4S,5R,6R)-3,5-dihydroxy-6-(hydroxymethyl)-4-[(3R,4S,5S,6R)-3,4,5-trihydroxy-6-(hydroxymethyl) oxan-2-yl]oxyoxan-2-yl]oxy-6-(hydroxymethyl) oxane-2,3,5-triol, (3R,6S)-3-hydroxy-3-[(3-hydroxyphenyl)methyl]-1,4-dimethyl-6-[(4-nitro-1H-indol-3-yl)methyl]piperazine-2,5-dione, 2,4-dichloro-6-[(3-methoxyphenyl)iminomethyl]phenol, 1,2-benzothiazole 1,1-dioxide, 5-[(3-carboxy-4-hydroxyphenyl)-(3-carboxy-4-oxocyclohexa-2,5-dien-1-ylidene)methyl]-2-hydroxybenzoic acid, disodium 6-methyl-2-[4-[2-[4-(6-methyl-7-sulfonato-1,3-benzothiazol-2-yl)phenyl]iminohydrazinyl]phenyl]-1,3-benzothiazole-7-sulfonate, 8-[[4-methyl-3-[[3-[[3-[[2-methyl-5-[(4,6,8-trisulfonaphthalen-1-yl)carbamoyl]phenyl]carbamoyl]phenyl]carbamoylamino]benzoyl]amino]benzoyl]amino]nap hthalene-1,3,5-trisulfonic acid, 1-[4-Fluoro-3-(trifluoromethyl)phenyl]-3-(5-pyridin-4-yl-1,3,4-thiadiazol-2-yl) urea, 3-[18-(2-carboxyethyl)-7,12-diethyl-3,8, 13, 17,22-pentamethyl-23H-porphyrin-2-yl]propanoic acid, 1h-pyrrole-2,5-dione, 1-[(1-oxopropoxy)methyl]-N-propionyloxymethyl-maleimide, 3,4-dichloro-1-[3-(3,4-dichloro-2,5-dioxopyrrol-1-yl)-2,2-dimethylpropyl]pyrrole-2,5-dione, 5,7-dioxa-12-azoniapentacyclo[10.6.1.02,10.04,8.015,19]nonadeca-1 (18),2,4 (8),9,11,15 (19), 16-heptaen-17-ol, 5,6-bis(((E)-benzylidene)amino)-2-thioxo-2,3-dihydropyrimidin-4 (1H)-one, (6Z)-3,7,11-trimethyldodeca-1,6,10-trien-3-ol, (2S,5R)-5-methyl-2-propan-2-ylcyclohexan-1-one, (1S,2S,5S,8R,9S,10S,11R, 15S,18R)-9,10,15, 18-tetrahydroxy-12, 12-dimethyl-6-methylidene-17-oxapentacyclo[7.6.2.15,8.01, 11.02,8]octadecan-7-one, (1R,2R,5R,9R,12R,16R)-5-ethenyl-1,5, 12-trimethyl-10-oxatetracyclo[7.6.1.02,7.012,16]hexadec-7-ene-11,13-dione, (1S,2R,5R, 12R, 18R)-5-ethenyl-13-hydroxy-5,12-dimethyl-10,14-dioxapentacyclo[11.2.2.11,9.02,7.012,18]octadec-7-en-11-one, [(1R,2R,4S,6S,9R, 10S, 13S, 16R)-2, 16-dihydroxy-5,5,9-trimethyl-14-methylidene-15-oxo-6-tetracyclo[11.2.1.01, 10.04,9]hexadecanyl]acetate, (1S,4S,8R,9R,12S, 13S, 14S, 16S)-9, 14-dihydroxy-7,7-dimethyl-17-methylidene-3,10-dioxapentacyclo[14.2.1.01,13.04,12.08,12]nonadecane-2, 18-dione, [(1S,1′R,3'S,5R, 6S, 7S,9S)-3′-acetyloxy-7-hydroxy-6′,6′-dimethyl-10-methylidene-2,11-dioxospiro[3-oxatricyclo[7.2.1.01,6]dodecane-5,2′-cyclohexane]-1′-yl]methyl acetate, (3E,6E)-3,7,11-trimethyldodeca-1,3,6,10-tetraene, 6,6-dimethyl-2-methylidenebicyclo[3.1.1]heptane, 2,2-dimethyl-3-methylidenebicyclo[2.2.1]heptane, 5-methyl-2-propan-2-ylphenol, (2E)-3,7-dimethylocta-2,6-dien-1-ol, (2E)-3,7-dimethylocta-2,6-dienal, 5-methyl-2-propan-2-ylcyclohexan-1-ol, benzoic acid, 2-aminophenoxazin-3-one, 2-amino-7-methoxyphenoxazin-3-one, (2S,3R)-2-(3,4-dihydroxyphenyl)-3,4-dihydro-2H-chromene-3,5,7-triol, and any combination thereof.

47-55. (canceled)

56. The method of claim 35, wherein the compound that modulates meiotic recombination is present in a composition and applying the compound that modulates meiotic recombination to the plant comprises applying the composition to the plant; optionally wherein the composition is a liquid, optionally wherein the liquid comprises water, dimethylsulfoxide, and/or a surfactant; optionally wherein the compound that modulates meiotic recombination is present in the composition in an amount of about 0.001 or 0.01 mM to about 20 mM.

57.-58. (canceled)

59. The method of claim 35, wherein applying the compound that modulates meiotic recombination to the plant comprises:
applying the compound that modulates meiotic recombination to the plant at a time prior to, during, and/or after the transition from the vegetative phase to the reproductive phase of the plant, optionally wherein the compound is applied to the plant during the plant's reproductive phase;
applying the compound that modulates meiotic recombination to a cell of the plant, wherein the cell is a meiocyte and/or is undergoing meiosis, optionally wherein the cell is a vegetative cell;
applying the compound that modulates meiotic recombination to the sporophyte generation of the plant to modulate meiotic recombination in the gametophyte generation, optionally wherein the gametophyte generation has an increased number of crossovers compared to the gametophyte generation generated in the absence of a method of the present invention and/or to the parent generation generated in the absence of a method of the present invention;
applying the compound that modulates meiotic recombination to a reproductive part of the plant (e.g., flower bud, inflorescence, flower, stamen, pistil, etc.); and/or
applying the compound that modulates meiotic recombination to the aerial parts of the plant.

60.-63. (canceled)

64. The method of claim 35, wherein the method reduces linkage drag in the plant and/or its progeny.
